(19) United States
(12) Patent Application Publication
(10) Pub. No.: US 2023/0190199 A1
(43) Pub. Date: Jun. 22, 2023

(54) BED HAVING FEATURES TO PASSIVELY MONITOR BLOOD PRESSURE

(71) Applicant: Sleep Number Corporation, Minneapolis, MN (US)

(72) Inventor: Gary N. Garcia Molina, Madison, WI (US)

(21) Appl. No.: 18/084,944

(22) Filed: Dec. 20, 2022

Related U.S. Application Data
(60) Provisional application No. 63/352,394, filed on Jun. 15, 2022.
(60) Provisional application No. 63/292,928, filed on Dec. 22, 2021.

(51) Int. Cl. A61B 5/00; A61B 5/11; A61B 5/021

(52) U.S. Cl. CPC A61B 5/6892; A61B 5/1102; A61B 5/02125; A61B 5/6801; A61B 5/7267; A61B 5/4812; A61B 2562/04; A61B 2562/0252; A61B 2562/0247; A61B 2560/0223; A47C 27/10

(57) ABSTRACT

A bed has a mattress to support a user laying on the bed. A first balistocardiograph (BCG) sensor is configured to collect first-BCG data from a first location of the user laying on the bed due to pressure applied to the bed by the user. A second BCG sensor is configured to collect second-BCG data from a second location of the user. A computer-system may include a process and memory, the computer-system configured to: receive the first BCG-data and the second-BCG data and determine one or more blood-pressure (BP) values for the user.

### CROSS-REFERENCE TO RELATED APPLICATIONS

This application claims the benefit of U.S. Provisional Application Ser. No. 63/352,394, filed Jun. 15, 2022 and U.S. Provisional Application Ser. No. 63/292,928, filed Dec. 22, 2021. The disclosures of the prior applications are considered part of (and is incorporated by reference in) the disclosure of this application.

The present document relates to a consumer device such as an airbed having sensors and computing hardware.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

A bed can be used to passively monitor the blood pressure (BP) of a person on the bed. Using differential measure of cardiac activity, such as the time-difference of a pulse of blood to reach different areas of the body, a BP value for the person can be determined. This can include calculating a pulse transit time (PTT) using the signals of two or more balistocardiographic data sources for the person, and then using that PTT as an input to a model that uses PTT to estimate BP.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a bed having a mattress to support a user laying on the bed. The system also includes a first balistocardiograph (BCG) sensor configured to collect first-BCG data from a first location of the user laying on the bed due to pressure applied to the bed by the user. The system also includes a second BCG sensor configured to collect second-BCG data from a second location of the user. The system also includes a computer-system may include a process and memory, the computer-system configured to: receive the first BCG-data and the second-BCG data and determine one or more blood-pressure (BP) values for the user. Other embodiments of this aspect include corresponding computer systems, apparatus, and computer programs recorded on one or more computer storage devices, each configured to perform the actions of the methods.

Implementations may include one or more of the following features. The system where the first BCG sensor is a pressure sensor configured to sense pressure readings applied to the bed by the user due to weight and motion of the user. The first BCG sensor is one of the group may include of a pressure transducer and a load cell. The second BCG sensor is a system may include one or more load cells configured to sense load applied to a support member of the bed by the user due to weight and motion of the user. The second BCG sensor is a device worn by the user on a specified location of the user's body. The second BCG sensor is a camera configured to sense energy emissions of the user's skin, the energy emissions being one of the group may include of visible light and non-visible energy. To determine one or more BP values for the user, the computer-system is configured to: determine a pulse transit time (PTT) for the user identifying a length of time between a pulse event in the first BCG data and the second BCG data representing the length of time between when a pulse of the user's blood reaches the first location and the second location; providing, as input, the PTT to a BP classifier; and receiving, as output, the one or more blood-pressure values for the user. The BP classifier has been trained using at least one model of the group may include of: i) a linear model finding a fit between training-PTT values and training-BP values; ii) a polynomial model finding coefficients describing a function that relates the training-PTT values with the training-BP value; iii) a machine-learning model that creates a data-structure created by a machine-learning process. The data structure is a convolutional neural network. The computer-system is further configured to: identify a time window of a particular sleep stage of a sleep session of the user sleeping on the bed; identify a representative-BP for the sleep stage based on the one or more blood-pressure (BP) values for the user which are within the time window. The representative-BP is the nth lowest BP value within the time window to represent a low-BP value for the sleep stage. The representative-BP is the nth lowest percentile BP value. The computer system is further configured to, responsive to determining the BP-dip value, perform, using the BP-dip value, at least one of the group may include of: storing the BP-dip to the memory, generating an alert for output to a user-output device, engaging an automated device. The computer-system is further configured to: receive an instant-BP value for the user taken after the sleep session; comparing the low-BP value for the sleep stage with the instant-BP value to determine a BP-dip value for the user representing an amount of dip in BP the user demonstrates in the sleep session. The time window is between 0 and 4 hours after sleep onset. The computer-system is further configured to, responsive to determining the BP value, perform, using the BP value, at least one of the group may include of: storing the BP value to the memory, generating an alert for output to a user-output device, engaging an automation device. Implementations of the described techniques may include hardware, a method or process, or computer software on a computer-accessible medium.

The devices, system, and techniques described herein may provide one or more of the following advantages. For example, a person can monitor their BP using passive sensing that the user does not need to engage or turn on. In some configurations, any time the user is on the bed, the system can automatically monitor BP. This means that the user does not need to remember or stick to a routine that they may have compliance issues with. In addition, this technology can be used for whom other types of at-home BP monitoring is not possible due to the conditions that calls for them to monitor their BP at home. For example, a bariatric patient who has a hard time finding an at-home inflatable cuff that fits can use this bed. Then, as the bariatric patient improves their condition and has a smaller arm, they would not need to find progressively smaller cuffs. In another instance, a healthy adult may develop hypertension, hypotension, or another condition such as a drug interaction that does not manifest with observable symptoms. As such, the person would know to check their BP. But because the bed can be used to passively monitor BP, instant (BP at a single moment in time) and trend (BP over a single sleep session or weeks and months of sleep sessions) can be provided to the user when they show a BP value outside of normal ranges.

This technology can work with bed and computing hardware purchased and used for other purposes. For example, a user may select a pressure adjustable air-bed that includes an air bladder, pressure sensor, load-cells, and controller for the added comfort that such a bed provides over other beds. But then, this hardware can perform double-duty, providing health monitoring without requiring more hardware than was already going to be in use. This can reduce costs and extend this functionality to those unable or unwilling to use single-purpose hardware. In some cases, the user can modify their bed once with a pressure-sensing pad or strip placed under a mattress and then not need to think about or separately store the hardware added to their bed.

This technology can be configured to use a minimum number of sensors, and also expandable to add more sensors if they are available. For example, in addition to the load cells and pressure sensors, the user may wear a wearable device like a watch or chest-strap, and those data sources can be incorporated into the BP sensing technology to increase accuracy and redundancy in case one sensing modality fails.

This technology can capture BP while the user is asleep, which may be otherwise accomplished with difficulty and in ways that could alter the user's BP as it is being monitored. For example, a user may suffer from White Coat Syndrome, in which anxiety about an illness or interaction with doctor can itself cause an acute increase in BP that could mask hypotension, cause a healthy BP to be classified as hypertension, and incorrectly increase the intensity of measured hypertension. For such a user, a night in a sleep study lab or hospital cannot provide an accurate measure of BP. Similarly, other systems for measuring sleeping BP can include contacting sensors that can disrupt sleep. For example, a cuff can be worn that inflates hourly to measure BP. However, especially for hypertensive patients, the pressure needed for the cuff to collect this information may be painful or distracting enough to interrupt the sleep session, which itself may increase BP. However, this technology can be created using nothing that contacts the user's body more than normal bed use—sleeping on a mattress with sheets and a blanket. Or the technology can be used with contacting, but not bothersome, contact such as a watch that happens to measure pulse. In doing so, this technology can avoid these limitations and be advantageously used by users not able to take advantage of other schemes for monitoring sleeping BP. Continuous BP measurements may be particularly advantageous for users (including patients being seen by medical providers) with BP disorders. A single measurement in these users can fail to provide the overall picture that the physician needs to recommend treatments. During sleep, there is the phenomenon known as BP-dipping; i.e., a drop in BP that occurs during sleep. Failure to dip or even reverse BP pattern is believed to be strongly associated with heart failure risk. This technology advantageously allows detection of abnormal BP changes (e.g. failure to dip) during sleep, enabling users and their care providers to identify and treat causes of BD-dipping or other patterns detectable with continuous BP measurements during sleep.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9 is a block diagram of an example of a sensory array that can be used in a data processing system associated with a bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19 is a diagram of a system with a bed used to passively monitor blood pressure.

FIG. 20 shows elements of pressure signals used to determine pulse transit times.

FIG. 21 is a swimlane diagram of an example process for determining blood pressure values.

FIG. 22 is a flowchart of an example process for determining blood pressure values.

FIG. 23 is a flowchart of an example process for determining a metric of risk based on blood pressure values.

FIG. 24 shows an example of two BCGs captured and plotted against a directly measured ECG

Like reference symbols in the various drawings indicate like elements.

### DETAILED DESCRIPTION

A bed can sense cardiac activity in, for example, two different points on the body. From this sensing, blood pressure (BP) can be determined, allowing a bed to be used as a passive BP sensor.

As blood travels through the body, a single contraction of the heart can transmit a pressure wave (i.e. the pulse) through the circulatory system. The speed of this transmission is believed to be a function of the pressure of the blood. Therefore, BP can be calculated by comparing when the pulse reaches one area of the body (e.g., the torso) with when it reaches another part of the body (e.g., a foot). Then, this time differential can be used with a function that transforms the time differential into BP or other similar measure.

Example Airbed Hardware

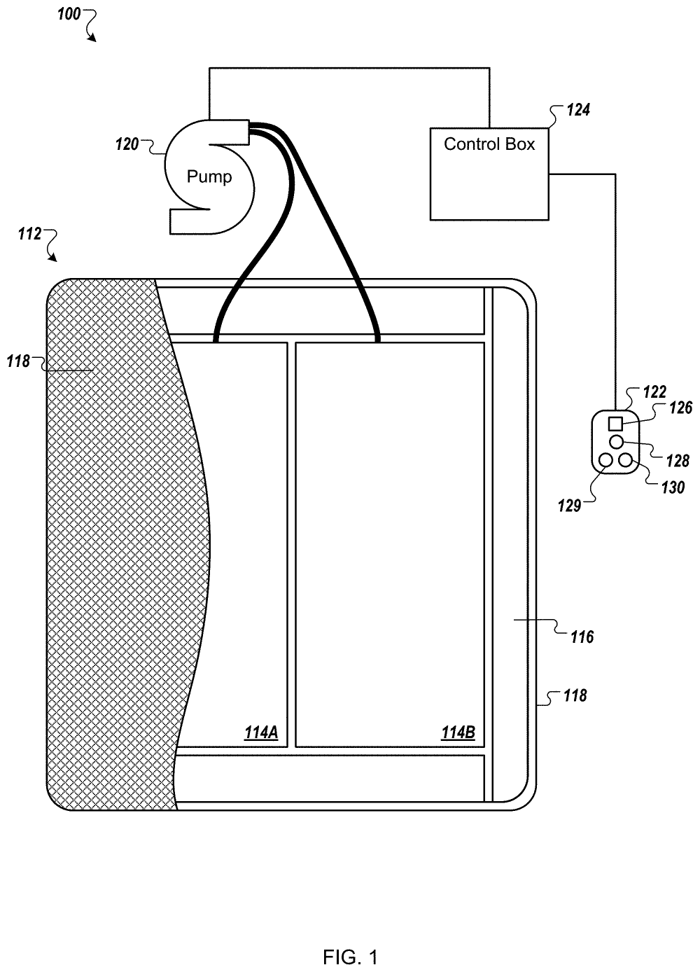

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application. For example, as mentioned above, the bed 112 can be controlled by a mobile device in wired or wireless communication with the bed 112.

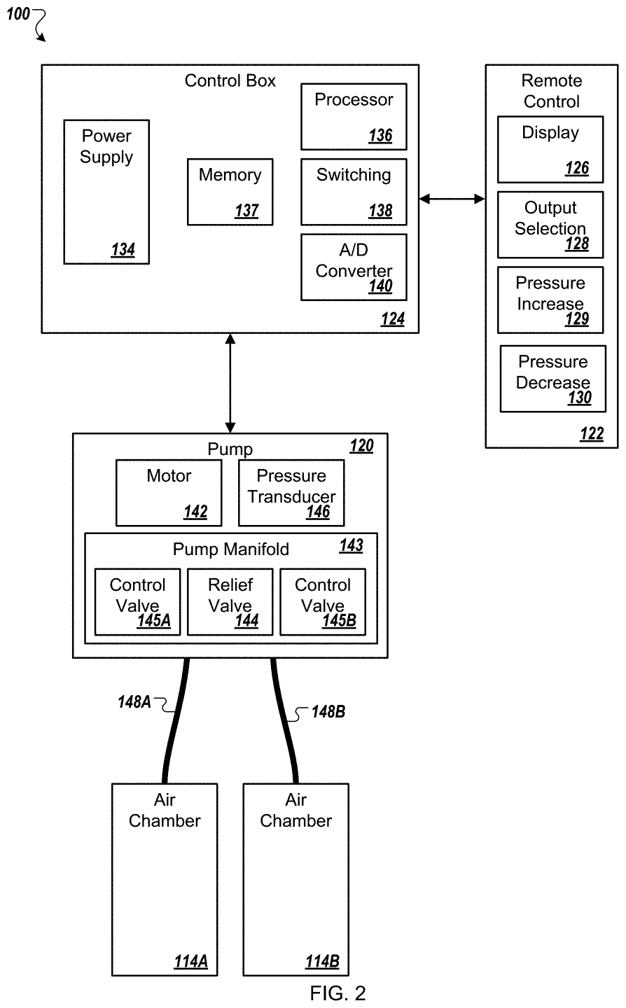

FIG. 2 is a block diagram of an example of various components of an air bed system. For example, these components can be used in the example air bed system 100. As shown in FIG. 2, the control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124.

The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. In yet some implementations, the control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The example air bed system 100 depicted in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber of the air bed system. As another example, a pump can be associated with multiple chambers of the air bed system. A first pump can, for example, be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Furthermore, additional pressure transducers can be incorporated into the air bed system 100 such that, for example, a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 in order to convey the pressure information to the user. The processor 136 can also send the digital signal to one or more other devices in wired or wireless communication with the air bed system, including but not limited to mobile devices such as smartphones, cellphones, tablets, computers, wearable devices, and home automation devices. As a result, the user can view pressure information associated with the air bed system at their mobile device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B in order to increase the firmness of the chamber, the pressure transducer 146 can sense pressure within the pump manifold 143. Again, the pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126 in order to convey the pressure information to the user.

Generally speaking, during an inflation or deflation process, the pressure sensed within the pump manifold 143 can provide an approximation of the pressure within the respective air chamber that is in fluid communication with the pump manifold 143. An example method of obtaining a pump manifold pressure reading that is substantially equivalent to the actual pressure within an air chamber includes turning off the pump 120, allowing the pressure within the air chamber 114A or 114B and the pump manifold 143 to equalize, and then sensing the pressure within the pump manifold 143 with the pressure transducer 146. Thus, providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers 114A and/or 114B. The pressure sensors can also be fluidly connected to the air chambers 114A and 114B, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user laying on the bed 112. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep stages) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes motion of the user, presence of the user on a surface of the bed 112, weight of the user, heart arrhythmia of the user, snoring of the user or another user on the air bed system, and apnea of the user. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence on the bed 112 can be beneficial to determine, by the processor 136, one or more adjustments to make to settings of the bed 112 (e.g., adjusting a firmness of the bed 112 when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present on the bed 112. As another example, the processor 136 can determine that the user is present on the bed 112 if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present on the bed 112. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed upon the bed.

In some implementations, fluctuations in pressure can be measured at the pump 120. For example, one or more pressure sensors can be located within one or more internal cavities of the pump 120 to detect fluctuations in pressure within the pump 120. The fluctuations in pressure detected at the pump 120 can indicate fluctuations in pressure in one or both of the chambers 114A and 114B. One or more sensors located at the pump 120 can be in fluid communication with one or both of the chambers 114A and 114B, and the sensors can be operative to determine pressure within the chambers 114A and 114B. The control box 124 can be configured to determine at least one vital sign (e.g., heartrate, respiratory rate) based on the pressure within the chamber 114A or the chamber 114B.

In some implementations, the control box 124 can analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of the force input applied to the chamber 114A from the user's movement, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can also receive additional biometric signals of the user from one or more other sensors or sensor arrays that are positioned on or otherwise integrated into the air bed system 100. For example, one or more sensors can be attached or removably attached to a top surface of the air bed system 100 and configured to detect signals such as heartrate, respiration rate, and/or motion of the user. The processor 136 can then combine biometric signals received from pressure sensors located at the pump 120, the pressure transducer 146, and/or the sensors positioned throughout the air bed system 100 to generate accurate and more precise heartrate, respiratory rate, and other information about the user and the user's sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's heartrate, respiratory rate, or other health information. The models can be trained using training data that includes training pressure signals and expected heartrates and/or respiratory rates. Sometimes, the control box 124 can determine the user's heartrate, respiratory rate, or other health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. Thus, the pressure signals generated by the pressure transducer 146 and received by the processor 136 can be filtered and indicated as corresponding to motion, heartbeat, or respiration. The processor 136 can also attribute such fluctuations in air pressure to sleep quality of the user. Such attributions can be determined based on applying one or more machine learning models and/or algorithms to the pressure signals generated by the pressure transducer 146. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the data collected by the pressure transducer 146 can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element that can be used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can therefore be selectively controlled by the temperature controller to provide cooling or heating that is preferred by each of the users on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of temperature controller, the pressure of the bed, the current position of the foundation or other information can be transmitted to control box 124. The control box 124 can then transmit the received information to the remote control 122, where the information can be displayed to the user (e.g., on the display 126). As described above, the control box 124 can also transmit the received information to a mobile device (e.g., smartphone, cellphone, laptop, tablet, computer, wearable device, or home automation device) to be displayed in a mobile application or other graphical user interface (GUI) to the user.

In some implementations, the example air bed system 100 further includes an adjustable foundation and an articulation controller configured to adjust the position of a bed (e.g., the bed 112) by adjusting the adjustable foundation that supports the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also be configured to provide different levels of massage to one or more portions of the bed 112 for one or more users on the bed 112. The user(s) can also adjust one or more massage settings for different portions of the bed 112 using the remote control 122 and/or a mobile device in communication with the air bed system 100, as described above.

Example of a Bed in a Bedroom Environment

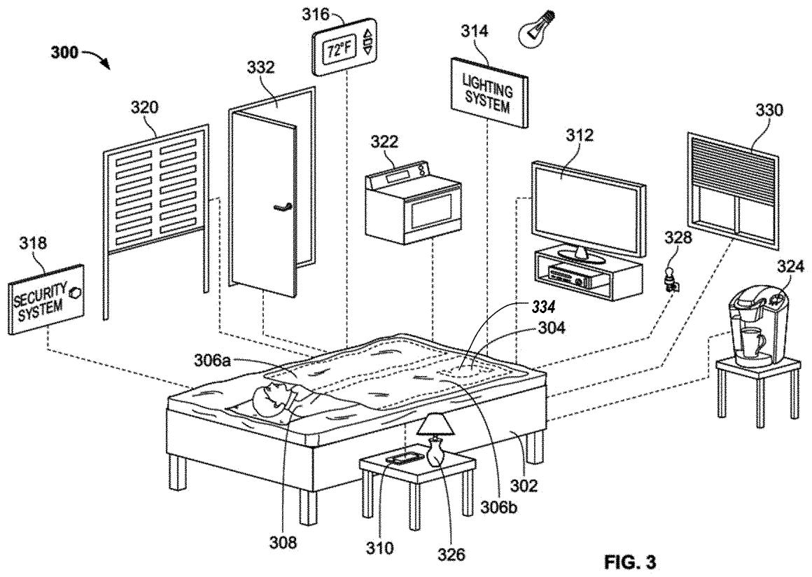

FIG. 3 shows an example environment 300 including a bed 302 in communication with devices located in and around a home. In the example shown, the bed 302 includes pump 304 for controlling air pressure within two air chambers 306a and 306b (as described above with respect to the air chambers 114A and 114B). The pump 304 additionally includes circuitry 334 for controlling inflation and deflation functionality performed by the pump 304. The circuitry 334 is further programmed to detect fluctuations in air pressure of the air chambers 306a-b and uses the detected fluctuations in air pressure to identify bed presence of a user 308, sleep state of the user 308, movement of the user 308, and biometric signals of the user 308, such as heartrate and respiration rate. The detected fluctuations in air pressure can also be used to detect when the user 308 is snoring and whether the user 308 has sleep apnea or other health conditions. Moreover, the detected fluctuations in air pressure can be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. As another example, the control circuitry 334 located within the pump 304 can communicate with control circuitry 334 at a remote location through a LAN or WAN (e.g., the internet). As yet another example, the control circuitry 334 can be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality and/or health related) about the user 308. For example, the bed 302 can include a second pump in addition to the pump 304, with each of the two pumps connected to a respective one of the air chambers 306a-b. For example, the pump 304 can be in fluid communication with the air chamber 306b to control inflation and deflation of the air chamber 306b as well as detect user signals for a user located over the air chamber 306b, such as bed presence, sleep state, movement, and biometric signals. The second pump can then be in fluid communication with the air chamber 306a and used to control inflation and deflation of the air chamber 306a as well as detect user signals for a user located over the air chamber 306a.

As another example, the bed 302 can include one or more pressure sensitive pads or surface portions that are operable to detect movement, including user presence, user motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located during sleep. The movement detected by the one or more pressure sensitive pads or surface portions can be used by control circuitry 334 to identify user sleep state, bed presence, or biometric signals for each of the users. The pressure sensitive pads can also be removable rather than incorporated into the surface of the bed 302.

The bed 302 can also include one or more temperature sensors and/or array of sensors that are operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and the user 308 falling asleep, total amount of time spent in the bed 302 for a given period of time, heartrate for the user 308 over a period of time, respiration rate for the user 308 over a period of time, or other information related to user interaction with the bed 302 by the user 308 or one or more other users of the bed 302. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308. For example, the information presented on the user device 310 can evolve with the age of the user 308 such that different information is presented on the user device 310 as the user 308 ages as a child or an adult.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to more accurately determine overall sleep quality and/or health of the user 308 based on information that is detected by one or more components (e.g., sensors) of the bed 302.

As another example, and as mentioned above, the user 308 can use the user device 310 as an interface for controlling air pressure of the air chambers 306a and 306b, for controlling various recline or incline positions of the bed 302, for controlling temperature of one or more surface temperature control devices of the bed 302, or for allowing the control circuitry 334 to generate control signals for other devices (as described in greater detail below).

In some implementations, the control circuitry 334 of the bed 302 can communicate with other devices or systems in addition to or instead of the user device 310. For example, the control circuitry 334 can communicate with the television 312, a lighting system 314, a thermostat 316, a security system 318, home automation devices, and/or other household devices, including but not limited to an oven 322, a coffee maker 324, a lamp 326, and/or a nightlight 328. Other examples of devices and/or systems that the control circuitry 334 can communicate with include a system for controlling window blinds 330, one or more devices for detecting or controlling the states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 of the bed 302 and other devices can occur through a network (e.g., a LAN or the Internet) or as point-to-point communication (e.g., using Bluetooth, radio communication, or a wired connection). In some implementations, control circuitry 334 of different beds 302 can communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. In some implementations, the control signals are generated based on information collected by the control circuitry 334, including information related to user interaction with the bed 302 by the user 308 and/or one or more other users. Information collected from one or more other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for one or more other devices, the control circuitry 334 can provide collected information (e.g., information related to user movement, bed presence, sleep state, or biometric signals for the user 308) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and one or more other devices in the house. The central controller can include one or more sensors that detect signals that can be used by the control circuitry 334 and/or the central controller to determine information about the user 308 (e.g., biometric or other health data, sleep quality, etc.). The sensors can detect signals including but not limited to ambient light, temperature, humidity, volatile organic compound(s), pulse, motion, and audio. These signals can be combined with signals that are detected by sensors of the bed 302 to determine more accurate information about the user 308's health and sleep quality. The central controller can provide controls (e.g., user-defined, presets, automated, user initiated, etc.) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that can output information and also receive input from the user 308. The display can output information such as the user 308's health, sleep quality, weather information, security integration features, lighting integration features, heating and cooling integration features, and other controls to automate devices in the house. The central controller can therefore operate to provide the user 308 with functionality and control of multiple different types of devices in the house as well as the user 308's bed 302.

Still referring to FIG. 3, the control circuitry 334 of the bed 302 can generate control signals for controlling actions of other devices, and transmit the control signals to the other devices in response to information collected by the control circuitry 334, including bed presence of the user 308, sleep state of the user 308, and other factors. For example, the control circuitry 334 integrated with the pump 304 can detect a feature of a mattress of the bed 302, such as an increase in pressure in the air chamber 306b, and use this detected increase in air pressure to determine that the user 308 is present on the bed 302. In some implementations, the control circuitry 334 can identify a heartrate or respiratory rate for the user 308 to identify that the increase in pressure is due to a person sitting, laying, or otherwise resting on the bed 302, rather than an inanimate object (such as a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experiencing improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 is able to use collected information (including information related to user interaction with the bed 302 by the user 308, as well as environmental information, time information, and input received from the user 308) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 pm and 11:00 pm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

For example, given the above example user bed presence, sleep, and wake patterns for the user 308, if the user 308 is detected as being on the bed 302 at 3:00 pm, the control circuitry 334 can determine that the user 308's presence on the bed 302 is only temporary, and use this determination to generate different control signals than would be generated if the control circuitry 334 determined that the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 has gotten out of bed at 3:00 am, the control circuitry 334 can use identified patterns for the user 308 to determine that the user has only gotten up temporarily (e.g., to use the bathroom, or get a glass of water) and is not up for the day. For example, the control circuitry 334 can turn on underbed lighting to assist the user 308 in carefully moving around the bed 302 and the room. By contrast, if the control circuitry 334 identifies that the user 308 has gotten out of the bed 302 at 6:40 am, the control circuitry 334 can determine that the user 308 is up for the day and generate a different set of control signals than those that would be generated if it were determined that the user 308 were only getting out of bed temporarily (as would be the case when the user 308 gets out of the bed 302 at 3:00 am) (e.g., the control circuitry 334 can turn on light 326 near the bed 302 and/or raise the window blinds 330 when it is determined that the user 308 is up for the day). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

As described above, the control circuitry 334 for the bed 302 can generate control signals for control functions of various other devices. The control signals can be generated, at least in part, based on detected interactions by the user 308 with the bed 302, as well as other information including time, date, temperature, etc. The control circuitry 334 can communicate with the television 312, receive information from the television 312, and generate control signals for controlling functions of the television 312. For example, the control circuitry 334 can receive an indication from the television 312 that the television 312 is currently turned on. If the television 312 is located in a different room than the bed 302, the control circuitry 334 can generate a control signal to turn the television 312 off upon making a determination that the user 308 has gone to bed for the evening or otherwise is remaining in the room with the bed 302. For example, if presence of the user 308 is detected on the bed 302 during a particular time range (e.g., between 8:00 pm and 7:00 am) and persists for longer than a threshold period of time (e.g., 10 minutes), the control circuitry 334 can determine that the user 308 is in bed for the evening. If the television 312 is on (as indicated by communications received by the control circuitry 334 of the bed 302 from the television 312), the control circuitry 334 can generate a control signal to turn the television 312 off. The control signals can be transmitted to the television (e.g., through a directed communication link between the television 312 and the control circuitry 334 or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 has indicated a preference for watching the morning news upon getting out of bed). The control circuitry 334 can generate the control signal and transmit the signal to the television 312 to cause the television 312 to turn on and tune to the desired station (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening. In response to this determination, the control circuitry 334 can generate control signals to cause lights in one or more rooms other than the room in which the bed 302 is located to switch off. The control signals can then be transmitted to the lighting system 314 and executed by the lighting system 314 to cause the lights in the indicated rooms to shut off. For example, the control circuitry 334 can generate and transmit control signals to turn off lights in all common rooms, but not in other bedrooms. As another example, the control signals generated by the control circuitry 334 can indicate that lights in all rooms other than the room in which the bed 302 is located are to be turned off, while one or more lights located outside of the house containing the bed 302 are to be turned on, in response to determining that the user 308 is in bed for the evening. Additionally, the control circuitry 334 can generate and transmit control signals to cause the nightlight 328 to turn on in response to determining user 308 bed presence or that the user 308 is asleep. As another example, the control circuitry 334 can generate first control signals for turning off a first set of lights (e.g., lights in common rooms) in response to detecting user bed presence, and second control signals for turning off a second set of lights (e.g., lights in the room in which the bed 302 is located) in response to detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also be configured to implement a sunrise lighting scheme when the user 308 wakes up in the morning. The control circuitry 334 can determine that the user 308 is awake for the day, for example, by detecting that the user 308 has gotten off of the bed 302 (e.g., is no longer present on the bed 302) during a specified time frame (e.g., between 6:00 am and 8:00 am). As another example, the control circuitry 334 can monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. For example, the sunrise lighting scheme can include lighting the bedroom with blue light to gently assist the user 308 in waking up and becoming active.

In some implementations, the control circuitry 334 can generate different control signals for controlling actions of one or more components, such as the lighting system 314, depending on a time of day that user interactions with the bed 302 are detected. For example, the control circuitry 334 can use historical user interaction information for interactions between the user 308 and the bed 302 to determine that the user 308 usually falls asleep between 10:00 pm and 11:00 pm and usually wakes up between 6:30 am and 7:30 am on weekdays. The control circuitry 334 can use this information to generate a first set of control signals for controlling the lighting system 314 if the user 308 is detected as getting out of bed at 3:00 am and to generate a second set of control signals for controlling the lighting system 314 if the user 308 is detected as getting out of bed after 6:30 am. For example, if the user 308 gets out of bed prior to 6:30 am, the control circuitry 334 can turn on lights that guide the user 308's route to a bathroom. As another example, if the user 308 gets out of bed prior to 6:30 am, the control circuitry 334 can turn on lights that guide the user 308's route to the kitchen (which can include, for example, turning on the nightlight 328, turning on under bed lighting, turning on the lamp 326, or turning on lights along a path that the user 308 takes to get to the kitchen).

As another example, if the user 308 gets out of bed after 6:30 am, the control circuitry 334 can generate control signals to cause the lighting system 314 to initiate a sunrise lighting scheme, or to turn on one or more lights in the bedroom and/or other rooms. In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach the bathroom, kitchen, or another destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can then identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). In some implementations, buffer time can be added to these timeframes. For example, if the user is identified as typically going to bed between 10:00 pm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 pm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of the year (e.g., earlier bed time during winter vs. summer) or at different times of the week (e.g., user 308 wakes up earlier on weekdays than on weekends).

The control circuitry 334 can distinguish between the user 308 going to bed for an extended period (such as for the night) as opposed to being present on the bed 302 for a shorter period (such as for a nap) by sensing duration of presence of the user 308 (e.g., by detecting pressure signals and/or temperature signals of the user 308 on the bed 302 by one or more sensors that are integrated into the bed 302). In some examples, the control circuitry 334 can distinguish between the user 308 going to bed for an extended period (such as for the night) as opposed to going to bed for a shorter period (such as for a nap) by sensing duration of sleep of the user 308. For example, the control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can also be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

In some examples, the control circuitry 334 can automatically determine the bed time range of the user 308 without requiring user inputs. In some examples, the control circuitry 334 can determine the bed time range of the user 308 automatically and in combination with user inputs (e.g., using one or more signals that are sensed by sensors of the bed 302 and/or the central controller described above). In some examples, the control circuitry 334 can set the bed time range directly according to user inputs. In some examples, the control circuitry 334 can associate different bed times with different days of the week. In each of these examples, the control circuitry 334 can control one or more components (such as the lighting system 314, the thermostat 316, the security system 318, the oven 322, the coffee maker 324, the lamp 326, and the nightlight 328), as a function of sensed bed presence and the bed time range.

The control circuitry 334 can additionally communicate with the thermostat 316, receive information from the thermostat 316, and generate control signals for controlling functions of the thermostat 316. For example, the user 308 can indicate user preferences for different temperatures at different times, depending on the sleep state or bed presence of the user 308. For example, the user 308 may prefer an environmental temperature of 72 degrees when out of bed, 70 degrees when in bed but awake, and 68 degrees when sleeping. The control circuitry 334 of the bed 302 can detect bed presence of the user 308 in the evening and determine that the user 308 is in bed for the night. In response to this determination, the control circuitry 334 can generate control signals to cause the thermostat 316 to change the temperature to 70 degrees. The control circuitry 334 can then transmit the control signals to the thermostat 316. Upon detecting that the user 308 is in bed during the bed time range or asleep, the control circuitry 334 can generate and transmit control signals to cause the thermostat 316 to change the temperature to 68. The next morning, upon determining that the user 308 is awake for the day (e.g., the user 308 gets out of bed after 6:30 am), the control circuitry 334 can generate and transmit control circuitry 334 to cause the thermostat to change the temperature to 72 degrees.

The control circuitry 334 can also determine control signals to be transmitted to the thermostat 316 based on maintaining improved or preferred sleep quality of the user 308. In other words, the control circuitry 334 can determine adjustments to the thermostat 316 that are not merely based on user-inputted preferences. For example, the control circuitry 334 can determine, based on historic sleep patterns and quality of the user 308 and by applying one or more machine learning models, that the user 308 experiences their best sleep when the bedroom is at 74 degrees. The control circuitry 334 can receive temperature signals from one or more devices and/or sensors in the bedroom indicating a temperature of the bedroom. When the temperature is below 74 degrees, the control circuitry 334 can determine control signals that cause the thermostat 316 to activate a heating unit in the house to raise the temperature to 74 degrees in the bedroom. When the temperature is above 74 degrees, the control circuitry 334 can determine control signals that cause the thermostat 316 to activate a cooling unit in the house to lower the temperature back to 74 degrees. Sometimes, the control circuitry 334 can also determine control signals that cause the thermostat 316 to maintain the bedroom within a temperature range that is intended to keep the user 308 in particular sleep states and/or transition to next preferred sleep states.

In some implementations, the control circuitry 334 can generate control signals to cause one or more heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. As yet another example, the user 308 can pre-program various times at which the temperature at the surface of the bed should be raised or lowered. For example, the user 308 can program the bed 302 to raise the surface temperature to 76 degrees at 10:00 pm, and lower the surface temperature to 68 degrees at 11:30 pm. As another example, one or more temperature sensors on the surface of the bed 302 can detect microclimates of the user 308 on the bed 302. When a detected microclimate of the user 308 drops below a predetermined threshold temperature, the control circuitry 334 can activate a heating element to raise the user 308's body temperature, thereby improving the user 308's comfortability, maintaining the user 308 in their sleep cycle, transitioning the user 308 to a next preferred sleep state, and/or otherwise maintaining or improving the user 308's sleep quality.

In some implementations, in response to detecting user bed presence of the user 308 and/or that the user 308 is asleep, the control circuitry 334 can cause the thermostat 316 to change the temperature in different rooms to different values. For example, in response to determining that the user 308 is in bed for the evening, the control circuitry 334 can generate and transmit control signals to cause the thermostat 316 to set the temperature in one or more bedrooms of the house to 72 degrees and set the temperature in other rooms to 67 degrees. Other control signals are also possible, and can be based on user preference and user input.

The control circuitry 334 can also receive temperature information from the thermostat 316 and use this temperature information to control functions of the bed 302 or other devices. For example, as discussed above, the control circuitry 334 can adjust temperatures of heating elements included in or otherwise attached to the bed 302 (e.g., a foot warming pad) in response to temperature information received from the thermostat 316.

In some implementations, the control circuitry 334 can generate and transmit control signals for controlling other temperature control systems. For example, in response to determining that the user 308 is awake for the day, the control circuitry 334 can generate and transmit control signals for causing floor heating elements to activate in the bedroom and/or in other rooms in the house. For example, the control circuitry 334 can cause a floor heating system in a master bedroom to turn on in response to determining that the user 308 is awake for the day. One or more of the control signals described herein that are determined by the control circuitry 334 can also be determined by the central controller described above.

The control circuitry 334 can additionally communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. The control circuitry 334 can then transmit the control signals to the security system 318 to cause the security system 318 to engage (e.g., turning on security cameras along a perimeter of the house, automatically locking doors in the house, etc.). As another example, the control circuitry 334 can generate and transmit control signals to cause the security system 318 to disable in response to determining that the user 308 is awake for the day (e.g., user 308 is no longer present on the bed 302 after 6:00 am). In some implementations, the control circuitry 334 can generate and transmit a first set of control signals to cause the security system 318 to engage a first set of security features in response to detecting user bed presence of the user 308, and can generate and transmit a second set of control signals to cause the security system 318 to engage a second set of security features in response to detecting that the user 308 has fallen asleep.

In some implementations, the control circuitry 334 can receive alerts from the security system 318 and indicate the alert to the user 308. For example, the control circuitry 334 can detect that the user 308 is in bed for the evening and in response, generate and transmit control signals to cause the security system 318 to engage or disengage. The security system can then detect a security breach (e.g., someone has opened the door 332 without entering the security code, or someone has opened a window when the security system 318 is engaged). The security system 318 can communicate the security breach to the control circuitry 334 of the bed 302. In response to receiving the communication from the security system 318, the control circuitry 334 can generate control signals to alert the user 308 to the security breach. For example, the control circuitry 334 can cause the bed 302 to vibrate. As another example, the control circuitry 334 can cause portions of the bed 302 to articulate (e.g., cause the head section to raise or lower) in order to wake the user 308 and alert the user to the security breach. As another example, the control circuitry 334 can generate and transmit control signals to cause the lamp 326 to flash on and off at regular intervals to alert the user 308 to the security breach. As another example, the control circuitry 334 can alert the user 308 of one bed 302 regarding a security breach in a bedroom of another bed, such as an open window in a kid's bedroom. As another example, the control circuitry 334 can send an alert to a garage door controller (e.g., to close and lock the door). As another example, the control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller described above The central controller can then determine one or more responses to the security breach.

The control circuitry 334 can additionally generate and transmit control signals for controlling the garage door 320 and receive information indicating a state of the garage door 320 (e.g., open or closed). For example, in response to determining that the user 308 is in bed for the evening, the control circuitry 334 can generate and transmit a request to a garage door opener or another device capable of sensing if the garage door 320 is open. The control circuitry 334 can request information on the current state of the garage door 320. If the control circuitry 334 receives a response (e.g., from the garage door opener) indicating that the garage door 320 is open, the control circuitry 334 can either notify the user 308 that the garage door is open (e.g., by displaying a notification or other message at the user device 310, by outputting a notification at the central controller, etc.), and/or generate a control signal to cause the garage door opener to close the garage door 320. For example, the control circuitry 334 can send a message to the user device 310 indicating that the garage door is open. As another example, the control circuitry 334 can cause the bed 302 to vibrate. As yet another example, the control circuitry 334 can generate and transmit a control signal to cause the lighting system 314 to cause one or more lights in the bedroom to flash to alert the user 308 to check the user device 310 for an alert (in this example, an alert regarding the garage door 320 being open). Alternatively, or additionally, the control circuitry 334 can generate and transmit control signals to cause the garage door opener to close the garage door 320 in response to identifying that the user 308 is in bed for the evening and that the garage door 320 is open. Control signals can also vary depend on the age of the user 308.

The control circuitry 334 can similarly send and receive communications for controlling or receiving state information associated with the door 332 or the oven 322. For example, upon detecting that the user 308 is in bed for the evening, the control circuitry 334 can generate and transmit a request to a device or system for detecting a state of the door 332. Information returned in response to the request can indicate various states of the door 332 such as open, closed but unlocked, or closed and locked. If the door 332 is open or closed but unlocked, the control circuitry 334 can alert the user 308 to the state of the door, such as in a manner described above with reference to the garage door 320. Alternatively, or in addition to alerting the user 308, the control circuitry 334 can generate and transmit control signals to cause the door 332 to lock, or to close and lock. If the door 332 is closed and locked, the control circuitry 334 can determine that no further action is needed.

Similarly, upon detecting that the user 308 is in bed for the evening, the control circuitry 334 can generate and transmit a request to the oven 322 to request a state of the oven 322 (e.g., on or off). If the oven 322 is on, the control circuitry 334 can alert the user 308 and/or generate and transmit control signals to cause the oven 322 to turn off. If the oven is already off, the control circuitry 334 can determine that no further action is necessary. In some implementations, different alerts can be generated for different events. For example, the control circuitry 334 can cause the lamp 326 (or one or more other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., that a child of the user 308 has gotten out of bed in the middle of the night as sensed by a sensor in the child's bed). Other examples of alerts that can be processed by the control circuitry 334 of the bed 302 and communicated to the user (e.g., at the user device 310 and/or the central controller described herein) include a smoke detector detecting smoke (and communicating this detection of smoke to the control circuitry 334), a carbon monoxide tester detecting carbon monoxide, a heater malfunctioning, or an alert from any other device capable of communicating with the control circuitry 334 and detecting an occurrence that should be brought to the user 308's attention.

The control circuitry 334 can also communicate with a system or device for controlling a state of the window blinds 330. For example, in response to determining that the user 308 is in bed for the evening, the control circuitry 334 can generate and transmit control signals to cause the window blinds 330 to close. As another example, in response to determining that the user 308 is up for the day (e.g., user has gotten out of bed after 6:30 am) or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. As yet another example, the control circuitry 334 can generate and transmit control signals that cause a first set of blinds to close in response to detecting user bed presence of the user 308 and a second set of blinds to close in response to detecting that the user 308 is asleep.

The control circuitry 334 can generate and transmit control signals for controlling functions of other household devices in response to detecting user interactions with the bed 302. For example, in response to determining that the user 308 is awake for the day, the control circuitry 334 can generate and transmit control signals to the coffee maker 324 to cause the coffee maker 324 to begin brewing coffee. As another example, the control circuitry 334 can generate and transmit control signals to the oven 322 to cause the oven 322 to begin preheating (for users that like fresh baked bread in the morning or otherwise bake or prepare food in the morning). As another example, the control circuitry 334 can use information indicating that the user 308 is awake for the day along with information indicating that the time of year is currently winter and/or that the outside temperature is below a threshold value to generate and transmit control signals to cause a car engine block heater to turn on.

As another example, the control circuitry 334 can generate and transmit control signals to cause one or more devices to enter a sleep mode in response to detecting user bed presence of the user 308, or in response to detecting that the user 308 is asleep. For example, the control circuitry 334 can generate control signals to cause a mobile phone of the user 308 to switch into sleep mode or night mode such that notifications from the mobile phone are muted to not disturb the user 308's sleep. The control circuitry 334 can then transmit the control signals to the mobile phone. Later, upon determining that the user 308 is up for the day, the control circuitry 334 can generate and transmit control signals to cause the mobile phone to switch out of sleep mode.

In some implementations, the control circuitry 334 can communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302, etc.), the control circuitry 334 can generate and transmit control signals to cause one or more noise cancelation devices to activate. The noise cancelation devices can, for example, be included as part of the bed 302 or located in the bedroom with the bed 302. As another example, upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions with the bed 302. As mentioned throughout, functions of the bed 302 described herein can also be controlled by the user device 310 and/or the central controller (e.g., a hub device or other home automation device that controls multiple different devices in the home). As mentioned above, the bed 302 can include an adjustable foundation and an articulation controller configured to adjust the position of one or more portions of the bed 302 by adjusting the adjustable foundation that supports the bed 302. For example, the articulation controller can adjust the bed 302 from a flat position to a position in which a head portion of a mattress of the bed 302 is inclined upward (e.g., to facilitate a user sitting up in bed, reading, and/or watching television). In some implementations, the bed 302 includes multiple separately articulable sections. For example, portions of the bed corresponding to the locations of the air chambers 306a and 306b can be articulated independently from each other, to allow one person positioned on the bed 302 surface to rest in a first position (e.g., a flat position) while a second person rests in a second position (e.g., a reclining position with the head raised at an angle from the waist). In some implementations, separate positions can be set for two different beds (e.g., two twin beds placed next to each other). The foundation of the bed 302 can include more than one zone that can be independently adjusted. The articulation controller can also be configured to provide different levels of massage to one or more users on the bed 302 or to cause the bed to vibrate to communicate alerts to the user 308 as described above.

The control circuitry 334 can adjust positions (e.g., incline and decline positions for the user 308 and/or an additional user of the bed 302) in response to user interactions with the bed 302. For example, the control circuitry 334 can cause the articulation controller to adjust the bed 302 to a first recline position for the user 308 in response to sensing user bed presence for the user 308. The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the position of the bed 302 to a preferred user sleeping position (e.g., due to the user turning off the television 312 while the user 308 is in bed indicating that the user 308 wishes to go to sleep).

In some implementations, the control circuitry 334 can control the articulation controller so as to wake up one user of the bed 302 without waking another user of the bed 302. For example, the user 308 and a second user of the bed 302 can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located to wake the user 308 without disturbing the second user. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for, for example, engaging the security system 318, or instructing the lighting system 314 to turn off lights in various rooms, until both the user 308 and a second user are detected as being present on the bed 302. As another example, the control circuitry 334 can generate a first set of control signals to cause the lighting system 314 to turn off a first set of lights upon detecting bed presence of the user 308 and generate a second set of control signals for turning off a second set of lights in response to detecting bed presence of a second user. As another example, the control circuitry 334 can wait until it has been determined that both the user 308 and a second user are awake for the day before generating control signals to open the window blinds 330. As yet another example, in response to determining that the user 308 has left the bed 302 and is awake for the day, but that a second user is still sleeping, the control circuitry 334 can generate and transmit a first set of control signals to cause the coffee maker 324 to begin brewing coffee, to cause the security system 318 to deactivate, to turn on the lamp 326, to turn off the nightlight 328, to cause the thermostat 316 to raise the temperature in one or more rooms to 72 degrees, and/or to open the window blinds 330 in rooms other than the bedroom in which the bed 302 is located. Later, in response to detecting that the second user is no longer present on the bed (or that the second user is awake or is waking up) the control circuitry 334 can generate and transmit a second set of control signals to, for example, cause the lighting system 314 to turn on one or more lights in the bedroom, to cause window blinds in the bedroom to open, and to turn on the television 312 to a pre-specified channel. One or more other home automation control signals can be determined and generated by the control circuitry 334, the user device 310, and/or the central controller described herein.

Examples of Data Processing Systems Associated with a Bed

Described here are examples of systems and components that can be used for data processing tasks that are, for example, associated with a bed. In some cases, multiple examples of a particular component or group of components are presented. Some of these examples are redundant and/or mutually exclusive alternatives. Connections between components are shown as examples to illustrate possible network configurations for allowing communication between components. Different formats of connections can be used as technically needed or desired. The connections generally indicate a logical connection that can be created with any technologically feasible format. For example, a network on a motherboard can be created with a printed circuit board, wireless data connections, and/or other types of network connections. Some logical connections are not shown for clarity. For example, connections with power supplies and/or computer readable memory may not be shown for clarities sake, as many or all elements of a particular component may need to be connected to the power supplies and/or computer readable memory.

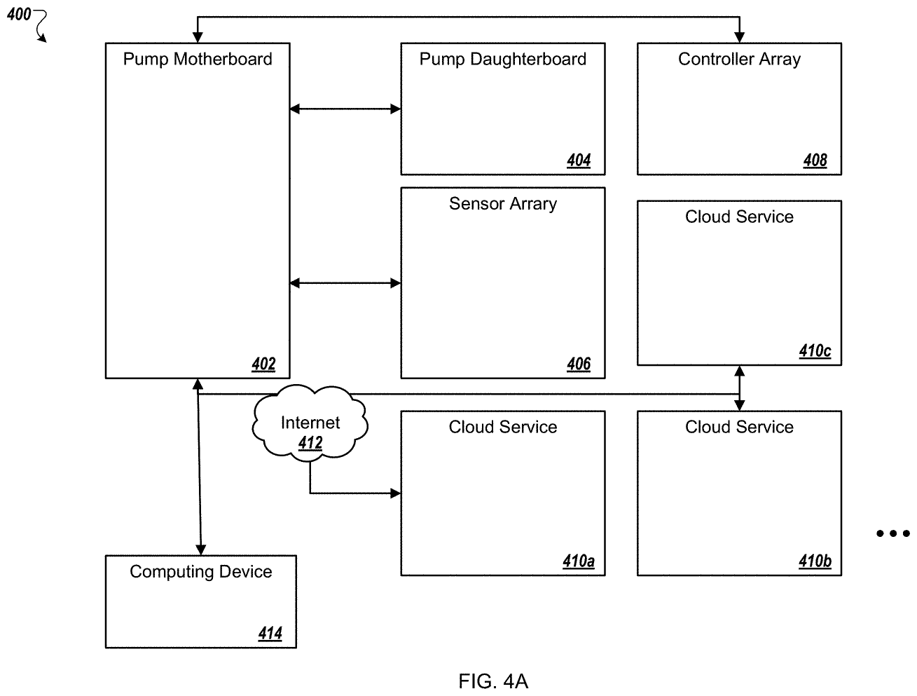

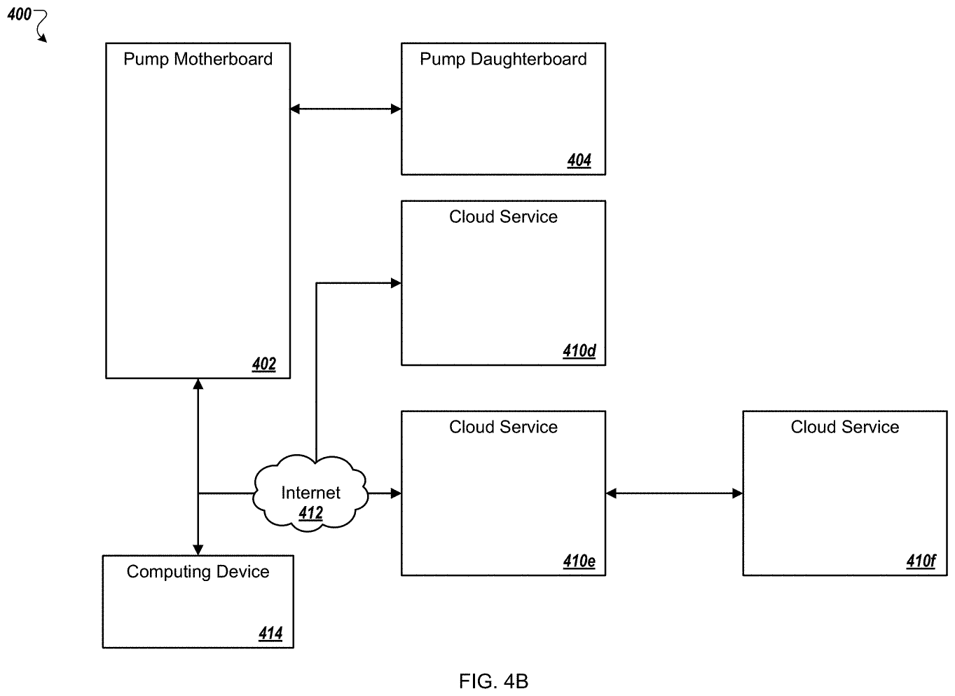

FIG. 4A is a block diagram of an example of a data processing system 400 that can be associated with a bed system, including those described above with respect to FIGS. 1-3. This system 400 includes a pump motherboard 402 and a pump daughterboard 404. The system 400 includes a sensor array 406 that can include one or more sensors configured to sense physical phenomenon of the environment and/or bed, and to report such sensing back to the pump motherboard 402 for, for example, analysis. The sensor array 406 can include one or more different types of sensors, including but not limited to pressure sensors, temperature sensors, light sensors, movement (e.g. motion) sensors, and audio sensors. The system 400 also includes a controller array 408 that can include one or more controllers configured to control logic-controlled devices of the bed and/or environment (such as home automation devices, security systems light systems, and other devices that are described in reference to FIG. 3). The pump motherboard 400 can be in communication with one or more computing devices 414 and one or more cloud services 410 over local networks, the Internet 412, or otherwise as is technically appropriate. Each of these components will be described in more detail, some with multiple example configurations, below.

In this example, a pump motherboard 402 and a pump daughterboard 404 are communicably coupled. They can be conceptually described as a center or hub of the system 400, with the other components conceptually described as spokes of the system 400. In some configurations, this can mean that each of the spoke components communicates primarily or exclusively with the pump motherboard 402. For example, a sensor of the sensor array 406 may not be configured to, or may not be able to, communicate directly with a corresponding controller. Instead, each spoke component can communicate with the motherboard 402. The sensor of the sensor array 406 can report a sensor reading to the motherboard 402, and the motherboard 402 can determine that, in response, a controller of the controller array 408 should adjust some parameters of a logic controlled device or otherwise modify a state of one or more peripheral devices. In one case, if the temperature of the bed is determined to be too hot based on received temperature signals from the sensor array 406, the pump motherboard 402 can determine that a temperature controller should cool the bed.

One advantage of a hub-and-spoke network configuration, sometimes also referred to as a star-shaped network, is a reduction in network traffic compared to, for example, a mesh network with dynamic routing. If a particular sensor generates a large, continuous stream of traffic, that traffic may only be transmitted over one spoke of the network to the motherboard 402. The motherboard 402 can, for example, marshal that data and condense it to a smaller data format for retransmission for storage in a cloud service 410. Additionally or alternatively, the motherboard 402 can generate a single, small, command message to be sent down a different spoke of the network in response to the large stream. For example, if the large stream of data is a pressure reading that is transmitted from the sensor array 406 a few times a second, the motherboard 402 can respond with a single command message to the controller array to increase the pressure in an air chamber of the bed. In this case, the single command message can be orders of magnitude smaller than the stream of pressure readings.

As another advantage, a hub-and-spoke network configuration can allow for an extensible network that can accommodate components being added, removed, failing, etc. This can allow, for example, more, fewer, or different sensors in the sensor array 406, controllers in the controller array 408, computing devices 414, and/or cloud services 410. For example, if a particular sensor fails or is deprecated by a newer version of the sensor, the system 400 can be configured such that only the motherboard 402 needs to be updated about the replacement sensor. This can allow, for example, product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. Then, for each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component will be further discussed. In some alternatives, two or more of the components of the system 400 can be realized in a single alternative component; some components can be realized in multiple, separate components; and/or some functionality can be provided by different components.

FIG. 4B is a block diagram showing some communication paths of the data processing system 400. As previously described, the motherboard 402 and the pump daughterboard 404 may act as a hub for peripheral devices and cloud services of the system 400. In cases in which the pump daughterboard 404 communicates with cloud services or other components, communications from the pump daughterboard 404 may be routed through the pump motherboard 402. This may allow, for example, the bed to have only a single connection with the internet 412. The computing device 414 may also have a connection to the internet 412, possibly through the same gateway used by the bed and/or possibly through a different gateway (e.g., a cell service provider).

Previously, a number of cloud services 410 were described. As shown in FIG. 4B, some cloud services, such as cloud services 410d and 410e, may be configured such that the pump motherboard 402 can communicate with the cloud service directly—that is the motherboard 402 may communicate with a cloud service 410 without having to use another cloud service 410 as an intermediary. Additionally or alternatively, some cloud services 410, for example cloud service 410f, may only be reachable by the pump motherboard 402 through an intermediary cloud service, for example cloud service 410e. While not shown here, some cloud services 410 may be reachable either directly or indirectly by the pump motherboard 402.

Additionally, some or all of the cloud services 410 may be configured to communicate with other cloud services. This communication may include the transfer of data and/or remote function calls according to any technologically appropriate format. For example, one cloud service 410 may request a copy for another cloud service's 410 data, for example, for purposes of backup, coordination, migration, or for performance of calculations or data mining. In another example, many cloud services 410 may contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

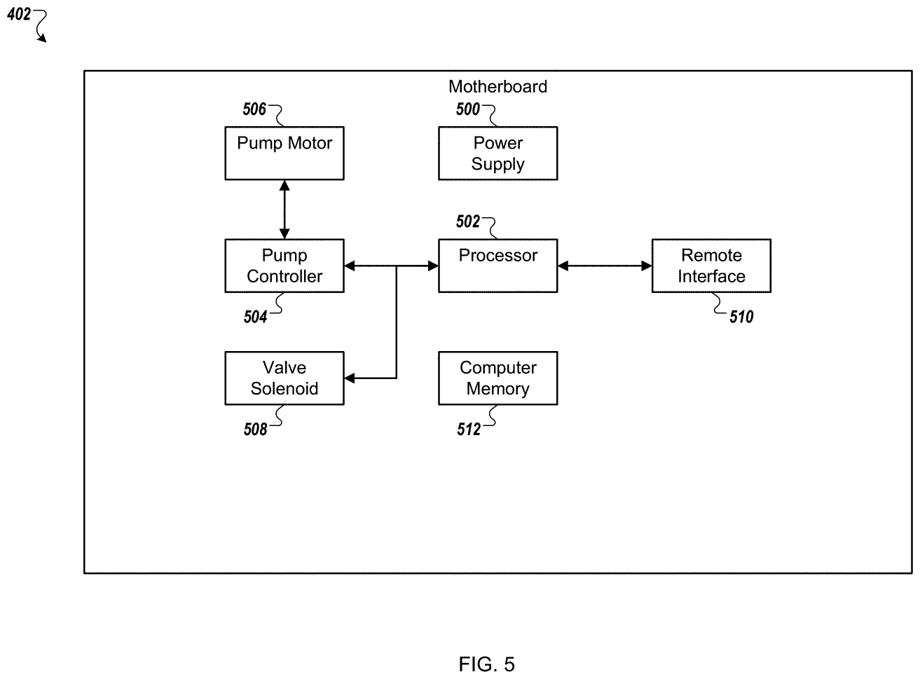

FIG. 5 is a block diagram of an example of a motherboard 402 that can be used in a data processing system that can be associated with a bed system, including those described above with respect to FIGS. 1-3. In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply can include, for example, a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware for performing the functionality needed.

The memory 512 is generally one or more devices for storing data. The memory 512 can include long term stable data storage (e.g., on a hard disk), short term unstable (e.g., on Random Access Memory) or any other technologically appropriate configuration.

The motherboard 402 includes a pump controller 504 and a pump motor 506. The pump controller 504 can receive commands from the processor 502 and, in response, control the functioning of the pump motor 506. For example, the pump controller 504 can receive, from the processor 502, a command to increase pressure of an air chamber by 0.3 pounds per square inch (PSI). The pump controller 504, in response, engages a valve so that the pump motor 506 is configured to pump air into the selected air chamber, and can engage the pump motor 506 for a length of time that corresponds to 0.3 PSI or until a sensor indicates that pressure has been increased by 0.3 PSI. In an alternative configuration, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

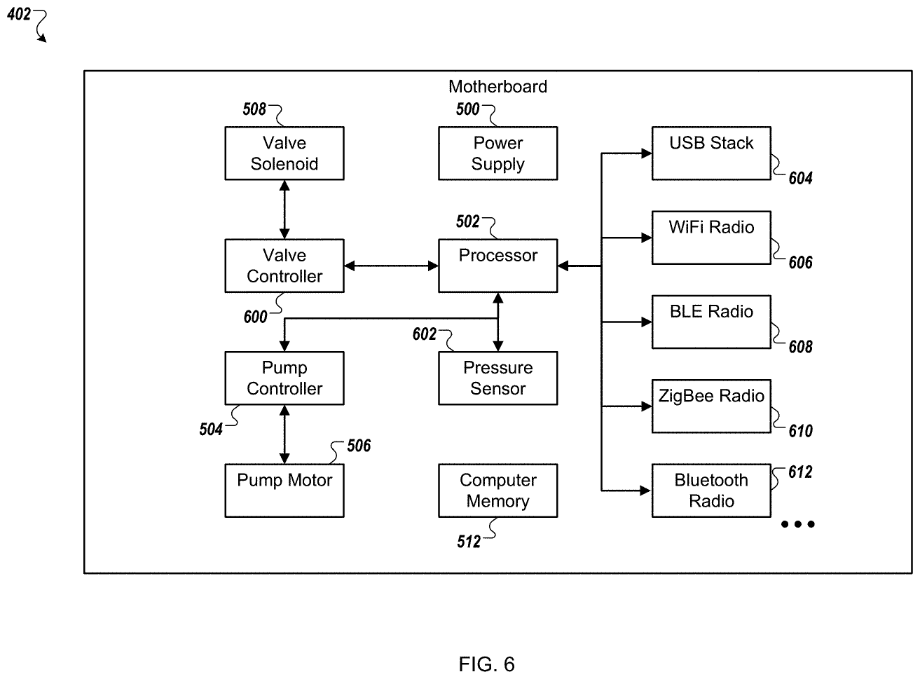

FIG. 6 is a block diagram of an example of the motherboard 402 that can be used in a data processing system associated with a bed system, including those described above with respect to FIGS. 1-3. Compared to the motherboard 402 described with reference to FIG. 5, the motherboard 402 in FIG. 6 can contain more components and provide more functionality in some applications.

In addition to the power supply 500, processor 502, pump controller 504, pump motor 506, and valve solenoid 508, this motherboard 402 is shown with a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

Similar to the way that the pump controller 504 converts commands from the processor 502 into control signals for the pump motor 506, the valve controller 600 can convert commands from the processor 502 into control signals for the valve solenoid 508. In one example, the processor 502 can issue a command to the valve controller 600 to connect the pump to a particular air chamber out of a group of air chambers in an air bed. The valve controller 600 can control the position of the valve solenoid 508 so that the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any number of devices, including but not limited to peripheral sensors, peripheral controllers, computing devices, and devices and services connected to the Internet 412.

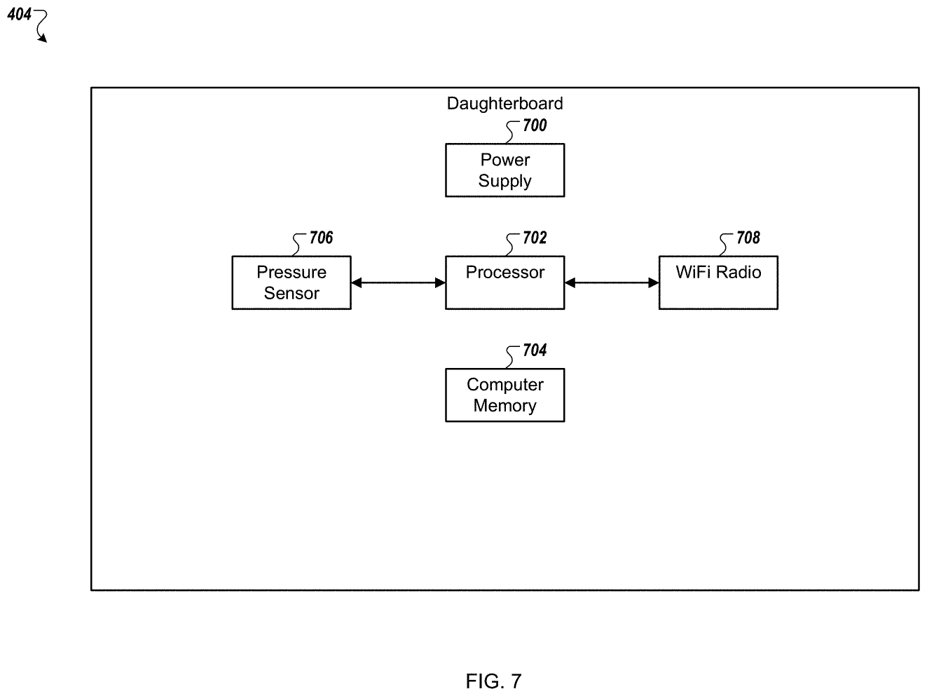

FIG. 7 is a block diagram of an example of a daughterboard 404 that can be used in a data processing system associated with a bed system, including those described above with respect to FIGS. 1-3. In some configurations, one or more daughterboards 404 can be connected to the motherboard 402. Some daughterboards 404 can be designed to offload particular and/or compartmentalized tasks from the motherboard 402. This can be advantageous, for example, if the particular tasks are computationally intensive, proprietary, or subject to future revisions. For example, the daughterboard 404 can be used to calculate a particular sleep data metric. This metric can be computationally intensive, and calculating the sleep metric on the daughterboard 404 can free up the resources of the motherboard 402 while the metric is being calculated. Additionally and/or alternatively, the sleep metric can be subject to future revisions. To update the system 400 with the new sleep metric, it is possible that only the daughterboard 404 that calculates that metric need be replaced. In this case, the same motherboard 402 and other components can be used, saving the need to perform unit testing of additional components instead of just the daughterboard 404.

The daughterboard 404 is shown with a power supply 700, a processor 702, computer readable memory 704, a pressure sensor 706, and a WiFi radio 708. The processor 702 can use the pressure sensor 706 to gather information about the pressure of an air chamber or chambers of an air bed. From this data, the processor 702 can perform an algorithm to calculate a sleep metric (e.g., sleep quality, whether a user is presently in the bed, whether the user has fallen asleep, a heartrate of the user, a respiration rate of the user, movement of the user, etc.). In some examples, the sleep metric can be calculated from only the pressure of air chambers. In other examples, the sleep metric can be calculated using signals from a variety of sensors (e.g., a movement sensor, a pressure sensor, a temperature sensor, and/or an audio sensor). In an example in which different data is needed, the processor 702 can receive that data from an appropriate sensor or sensors. These sensors can be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can then generate instructions for outputting the sleep metric to the user or otherwise using the sleep metric to determine one or more other information about the user or controls to control the bed system and/or peripheral devices.

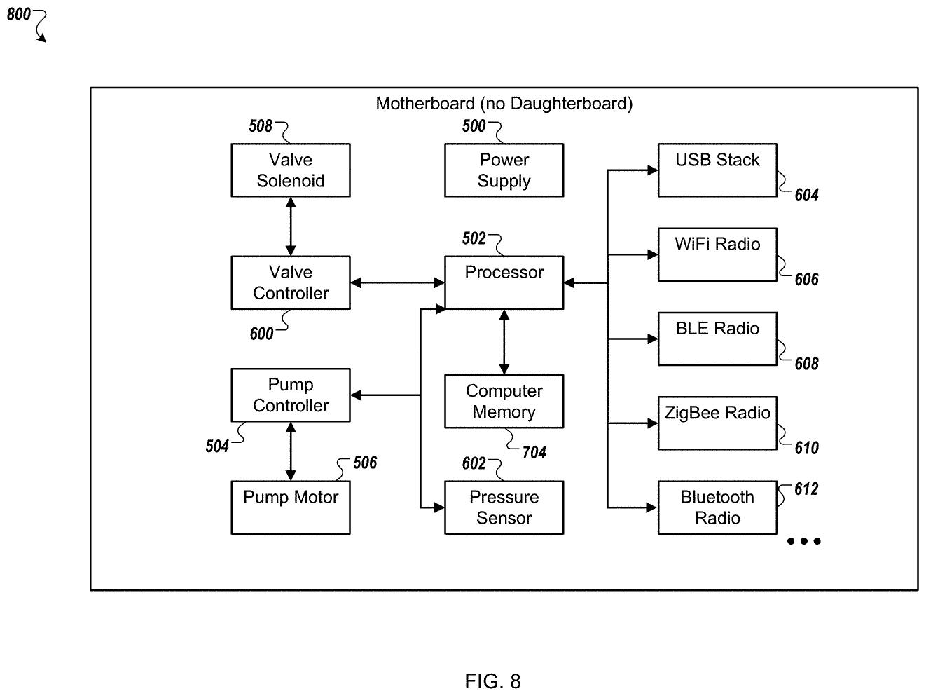

FIG. 8 is a block diagram of an example of a motherboard 800 with no daughterboard that can be used in a data processing system associated with a bed system, including those described above with respect to FIGS. 1-3. In this example, the motherboard 800 can perform most, all, or more of the features described with reference to the motherboard 402 in FIG. 6 and the daughterboard 404 in FIG. 7.

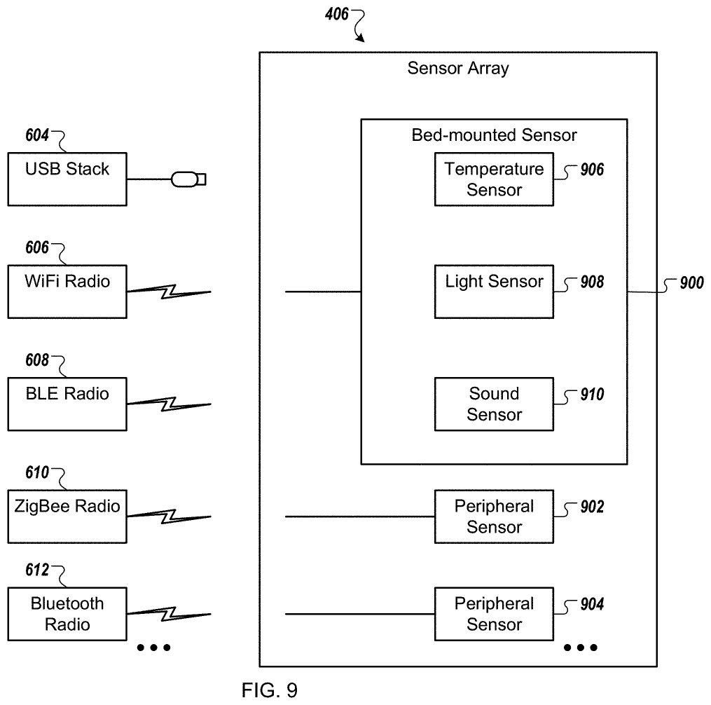

FIG. 9 is a block diagram of an example of the sensory array 406 that can be used in a data processing system associated with a bed system, including those described above with respect to FIGS. 1-3. In general, the sensor array 406 is a conceptual grouping of some or all the peripheral sensors that communicate with the motherboard 402 but are not native to the motherboard 402.

The peripheral sensors 902, 904, 906, 908, 910, etc. of the sensor array 406 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a sensor that outputs a reading over a USB cable can communicate through the USB stack 604.

Some of the peripheral sensors of the sensor array 406 can be bed mounted sensors 900, such as a temperature sensor 906, a light sensor 908, and a sound sensor 910. The bed mounted sensors 900 can be, for example, embedded into the structure of a bed and sold with the bed, or later affixed to the structure of the bed (e.g., part of a pressure sensing pad that is removably installed on a top surface of the bed, part of a temperature sensing or heating pad that is removably installed on the top surface of the bed, integrated into the top surface of the bed, attached along connecting tubes between a pump and air chambers, within air chambers, attached to a headboard of the bed, attached to one or more regions of an adjustable foundation, etc.). Other sensors 902 and 904 can be in communication with the motherboard 402, but optionally not mounted to the bed. The other sensors 902 and 904 can include a pressure sensor 902 and/or peripheral sensor 904. For example, the sensors 902 and 904 can be integrated or otherwise part of a user mobile device (e.g., mobile phone, wearable device, etc.). The sensors 902 and 904 can also be part of a central controller for controlling the bed and peripheral devices in the home. Sometimes, the sensors 902 and 904 can also be part of one or more home automation devices or other peripheral devices in the home.

In some cases, some or all of the bed mounted sensors 900 and/or sensors 902 and 904 can share networking hardware, including a conduit that contains wires from each sensor, a multi-wire cable or plug that, when affixed to the motherboard 402, connect all of the associated sensors with the motherboard 402. In some embodiments, one, some, or all of sensors 902, 904, 906, 908, and 910 can sense one or more features of a mattress, such as pressure, temperature, light, sound, and/or one or more other features of the mattress. In some embodiments, one, some, or all of sensors 902, 904, 906, 908, and 910 can sense one or more features external to the mattress. In some embodiments, pressure sensor 902 can sense pressure of the mattress while some or all of sensors 902, 904, 906, 908, and 910 can sense one or more features of the mattress and/or external to the mattress.

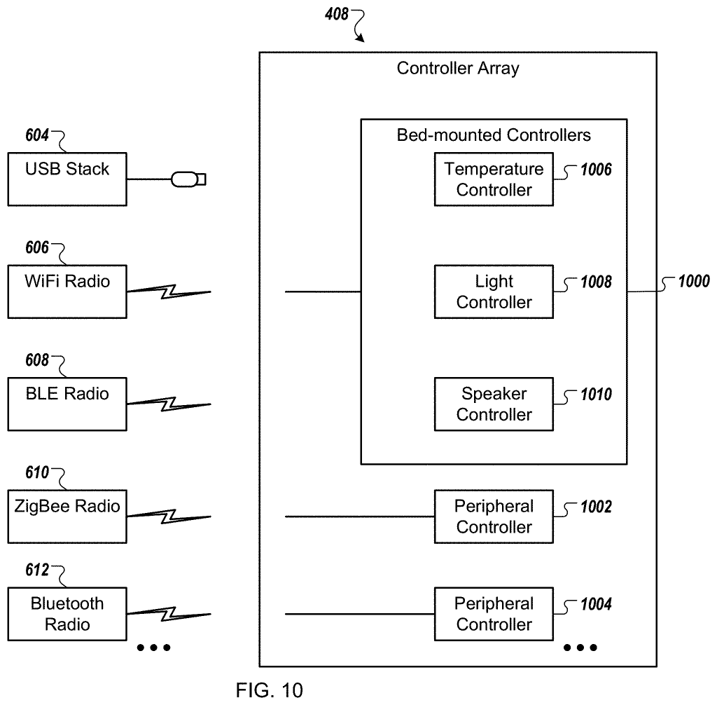

FIG. 10 is a block diagram of an example of the controller array 408 that can be used in a data processing system associated with a bed system, including those described above with respect to FIGS. 1-3. In general, the controller array 408 is a conceptual grouping of some or all peripheral controllers that communicate with the motherboard 402 but are not native to the motherboard 402.

The peripheral controllers of the controller array 408 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a controller that receives a command over a USB cable can communicate through the USB stack 604.

Some of the controllers of the controller array 408 can be bed mounted controllers 1000, such as a temperature controller 1006, a light controller 1008, and a speaker controller 1010. The bed mounting controllers 1000 can be, for example, embedded into the structure of a bed and sold with the bed, or later affixed to the structure of the bed, as described in reference to the peripheral sensors in FIG. 9. Other peripheral controllers 1002 and 1004 can be in communication with the motherboard 402, but optionally not mounted to the bed. In some cases, some or all of the bed mounted controllers 1000 and/or the peripheral controllers 1002 and 1004 can share networking hardware, including a conduit that contains wires for each controller, a multi-wire cable or plug that, when affixed to the motherboard 402, connects all of the associated controllers with the motherboard 402.

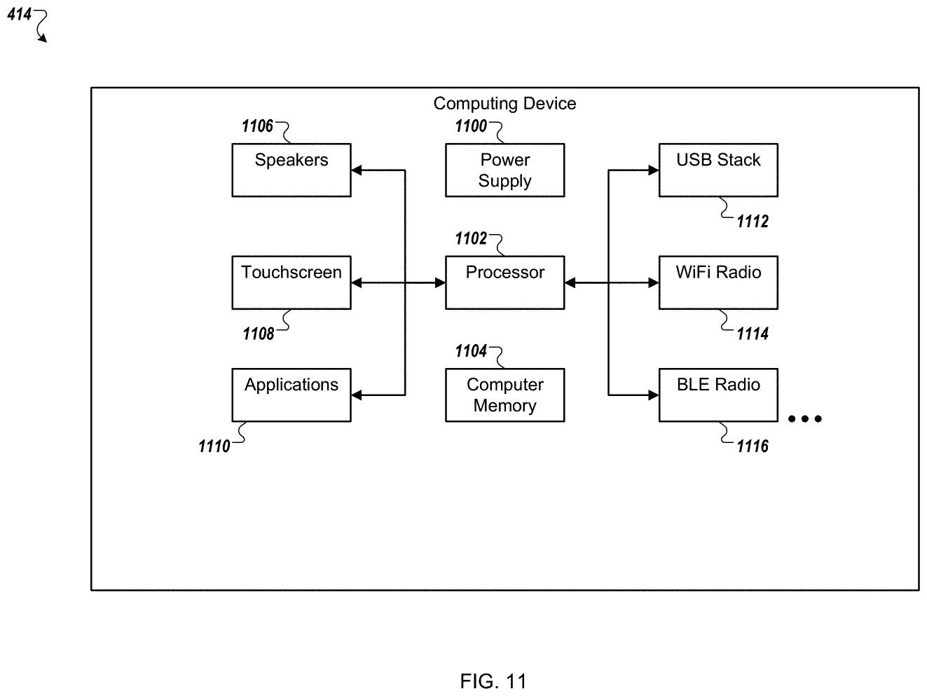

FIG. 11 is a block diagram of an example of the computing device 412 that can be used in a data processing system associated with a bed system, including those described above with respect to FIGS. 1-3. The computing device 412 can include, for example, computing devices used by a user of a bed. Example computing devices 412 include, but are not limited to, mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by, for example, speakers 1106, a touchscreen 1108, or other not shown components, such as a pointing device or keyboard. The computing device 412 can run one or more applications 1110. These applications can include, for example, applications to allow the user to interact with the system 400. These applications can allow a user to view information about the bed (e.g., sensor readings, sleep metrics), information about themselves (e.g., health conditions that are detected based on signals that are sensed at the bed), and/or configure the behavior of the system 400 (e.g., set a desired firmness to the bed, set desired behavior for peripheral devices). In some cases, the computing device 412 can be used in addition to, or to replace, the remote control 122 described previously.

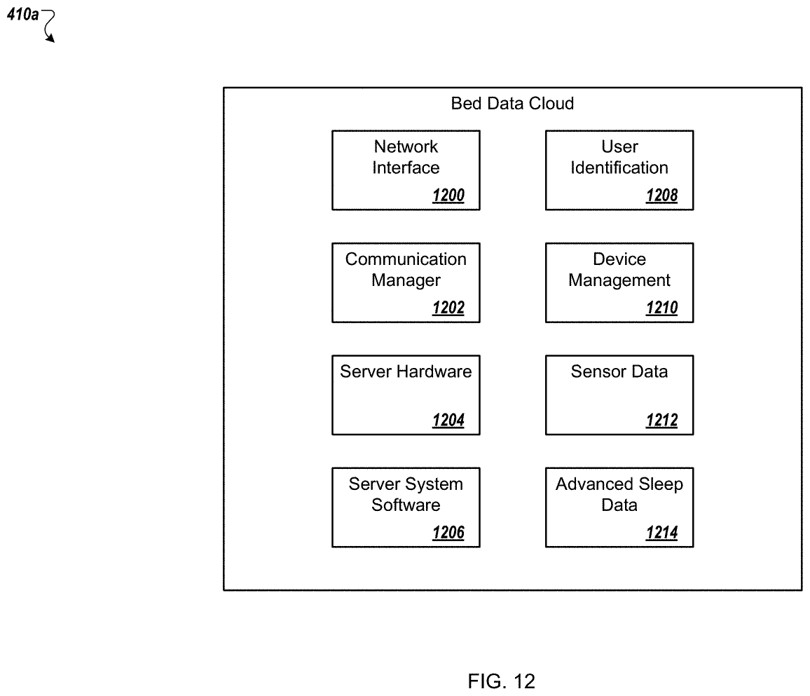

FIG. 12 is a block diagram of an example bed data cloud service 410a that can be used in a data processing system associated with a bed system, including those described above with respect to FIGS. 1-3. In this example, the bed data cloud service 410a is configured to collect sensor data and sleep data from a particular bed, and to match the sensor and sleep data with one or more users that use the bed when the sensor and sleep data was generated.

The bed data cloud service 410a is shown with a network interface 1200, a communication manager 1202, server hardware 1204, and server system software 1206. In addition, the bed data cloud service 410a is shown with a user identification module 1208, a device management 1210 module, a sensor data module 1210, and an advanced sleep data module 1214.

The network interface 1200 generally includes hardware and low level software used to allow one or more hardware devices to communicate over networks. For example the network interface 1200 can include network cards, routers, modems, and other hardware needed to allow the components of the bed data cloud service 410a to communicate with each other and other destinations over, for example, the Internet 412.

The communication manager 1202 generally comprises hardware and software that operate above the network interface 1200. This includes software to initiate, maintain, and tear down network communications used by the bed data cloud service 410a. This includes, for example, TCP/IP, SSL or TLS, Torrent, and other communication sessions over local or wide area networks. The communication manager 1202 can also provide load balancing and other services to other elements of the bed data cloud service 410a.

The server hardware 1204 generally includes physical processing devices used to instantiate and maintain the bed data cloud service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected.

The server system software 1206 generally includes software that runs on the server hardware 1204 to provide operating environments to applications and services. The server system software 1206 can include operating systems running on real servers, virtual machines instantiated on real servers to create many virtual servers, server level operations such as data migration, redundancy, and backup.

The user identification 1208 can include, or reference, data related to users of beds with associated data processing systems. For example, the users can include customers, owners, or other users registered with the bed data cloud service 410a or another service. Each user can have, for example, a unique identifier, user credentials, contact information, billing information, demographic information, or any other technologically appropriate information.

The device manager 1210 can include, or reference, data related to beds or other products associated with data processing systems. For example, the beds can include products sold or registered with a system associated with the bed data cloud service 410a. Each bed can have, for example, a unique identifier, model and/or serial number, sales information, geographic information, delivery information, a listing of associated sensors and control peripherals, etc. Additionally, an index or indexes stored by the bed data cloud service 410a can identify users that are associated with beds. For example, this index can record sales of a bed to a user, users that sleep in a bed, etc.

The sensor data 1212 can record raw or condensed sensor data recorded by beds with associated data processing systems. For example, a bed's data processing system can have a temperature sensor, pressure sensor, motion sensor, audio sensor, and/or light sensor. Readings from one or more of these sensors, either in raw form or in a format generated from the raw data (e.g. sleep metrics) of the sensors, can be communicated by the bed's data processing system to the bed data cloud service 410a for storage in the sensor data 1212. Additionally, an index or indexes stored by the bed data cloud service 410a can identify users and/or beds that are associated with the sensor data 1212.

The bed data cloud service 410a can use any of its available data, such as the sensor data 1212, to generate advanced sleep data 1214. In general, the advanced sleep data 1214 includes sleep metrics and other data generated from sensor readings, such as health information associated with the user of a particular bed. Some of these calculations can be performed in the bed data cloud service 410a instead of locally on the bed's data processing system, for example, because the calculations can be computationally complex or require a large amount of memory space or processor power that may not be available on the bed's data processing system. This can help allow a bed system to operate with a relatively simple controller and still be part of a system that performs relatively complex tasks and computations.

For example, the bed data cloud service 410a can retrieve one or more machine learning models from a remote data store and use those models to determine the advanced sleep data 1214. The bed data cloud service 410a can retrieve different types of models based on a type of the advanced sleep data 1214 that is being generated. As an illustrative example, the bed data cloud service 410a can retrieve one or more models to determine overall sleep quality of the user based on currently detected sensor data 1212 and/or historic sensor data (e.g., which can be stored in and accessed from a data store). The bed data cloud service 410a can retrieve one or more other models to determine whether the user is currently snoring based on the detected sensor data 1212. The bed data cloud service 410a can also retrieve one or more other models that can be used to determine whether the user is experiencing some health condition based on the detected sensor data 1212.

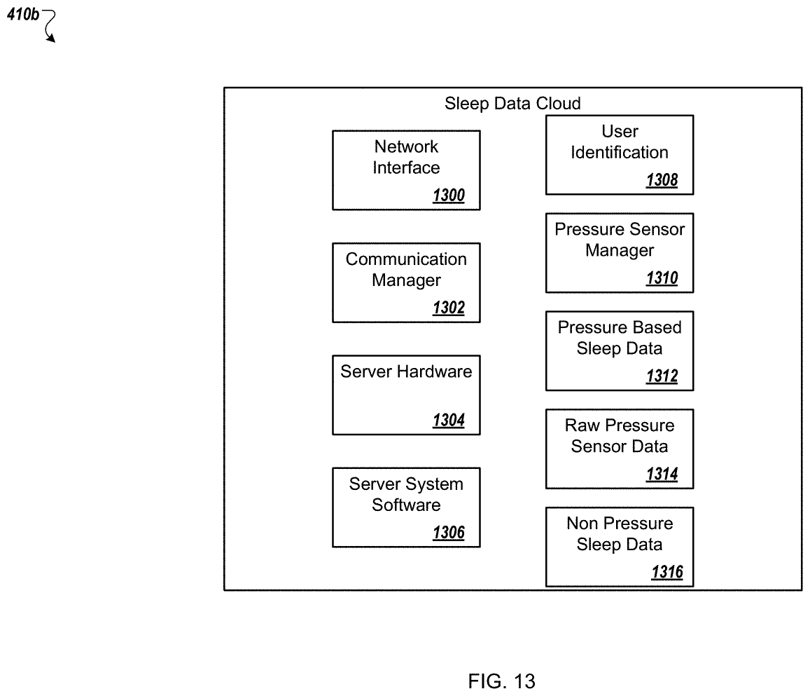

FIG. 13 is a block diagram of an example sleep data cloud service 410b that can be used in a data processing system associated with a bed system, including those described above with respect to FIGS. 1-3. In this example, the sleep data cloud service 410b is configured to record data related to users' sleep experience.

The sleep data cloud service 410b is shown with a network interface 1300, a communication manager 1302, server hardware 1304, and server system software 1306. In addition, the sleep data cloud service 410b is shown with a user identification module 1308, a pressure sensor manager 1310, a pressure based sleep data module 1312, a raw pressure sensor data module 1314, and a non-pressure sleep data module 1316. Sometimes, the sleep data cloud service 410b can include a sensor manager for each of the sensors that are integrated or otherwise in communication with the bed. In some implementations, the sleep data cloud service 410b can include a sensor manager that relates to multiple sensors in beds. For example, a single sensor manager can relate to pressure, temperature, light, movement, and audio sensors in a bed.

Referring to the sleep data cloud service 410b in FIG. 13, the pressure sensor manager 1310 can include, or reference, data related to the configuration and operation of pressure sensors in beds. For example, this data can include an identifier of the types of sensors in a particular bed, their settings and calibration data, etc.

The pressure based sleep data 1312 can use raw pressure sensor data 1314 to calculate sleep metrics specifically tied to pressure sensor data. For example, user presence, movements, weight change, heartrate, and breathing rate can all be determined from raw pressure sensor data 1314. Additionally, an index or indexes stored by the sleep data cloud service 410b can identify users that are associated with pressure sensors, raw pressure sensor data, and/or pressure based sleep data.

The non-pressure sleep data 1316 can use other sources of data to calculate sleep metrics. For example, user-entered preferences, light sensor readings, and sound sensor readings can all be used to track sleep data. Additionally, an index or indexes stored by the sleep data cloud service 410b can identify users that are associated with other sensors and/or non-pressure sleep data 1316.

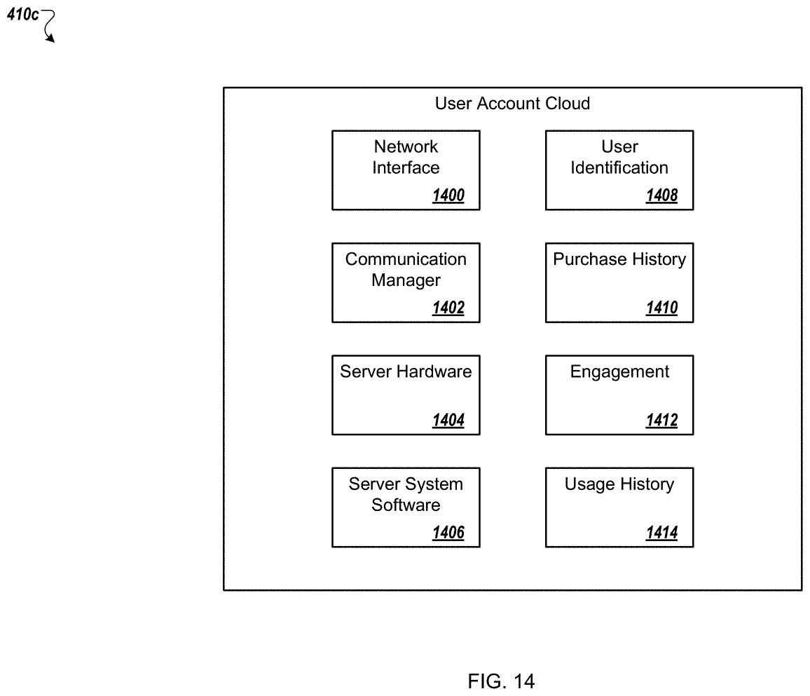

FIG. 14 is a block diagram of an example user account cloud service 410c that can be used in a data processing system associated with a bed system, including those described above with respect to FIGS. 1-3. In this example, the user account cloud service 410c is configured to record a list of users and to identify other data related to those users.

The user account cloud service 410c is shown with a network interface 1400, a communication manager 1402, server hardware 1404, and server system software 1406. In addition, the user account cloud service 410c is shown with a user identification module 1408, a purchase history module 1410, an engagement module 1412, and an application usage history module 1414.

The user identification module 1408 can include, or reference, data related to users of beds with associated data processing systems. For example, the users can include customers, owners, or other users registered with the user account cloud service 410c or another service. Each user can have, for example, a unique identifier, and user credentials, demographic information, or any other technologically appropriate information. Each user can also have user-inputted preferences pertaining to the user's bed system (e.g., firmness settings, heating/cooling settings, inclined and/or declined positions of different regions of the bed, etc.), ambient environment (e.g., lighting, temperature, etc.), and/or peripheral devices (e.g., turning on or off a television, coffee maker, security system, alarm clock, etc.).

The purchase history module 1410 can include, or reference, data related to purchases by users. For example, the purchase data can include a sale's contact information, billing information, and salesperson information that is associated with the user's purchase of the bed system. Additionally, an index or indexes stored by the user account cloud service 410c can identify users that are associated with a purchase of the bed system.

The engagement 1412 can track user interactions with the manufacturer, vendor, and/or manager of the bed and or cloud services. This engagement data can include communications (e.g., emails, service calls), data from sales (e.g., sales receipts, configuration logs), and social network interactions. The engagement data can also include servicing, maintenance, or replacements of components of the user's bed system.

The usage history module 1414 can contain data about user interactions with one or more applications and/or remote controls of a bed. For example, a monitoring and configuration application can be distributed to run on, for example, computing devices 412. The computing devices 412 can include a mobile phone, laptop, tablet, computer, smartphone, and/or wearable device of the user. The computing devices 412 can also include a central controller or hub device that can be used to control operations of the bed system and one or more peripheral devices. Moreover, the computing devices 412 can include a home automation device. The application that is presented to the user via the computing devices 412 can log and report user interactions for storage in the application usage history module 1414. Additionally, an index or indexes stored by the user account cloud service 410c can identify users that are associated with each log entry. User interactions that are stored in the application usage history module 1414 can optionally be used to determine or otherwise predict user preferences and/or settings for the user's bed and/or peripheral devices that can improve the user's overall sleep quality.

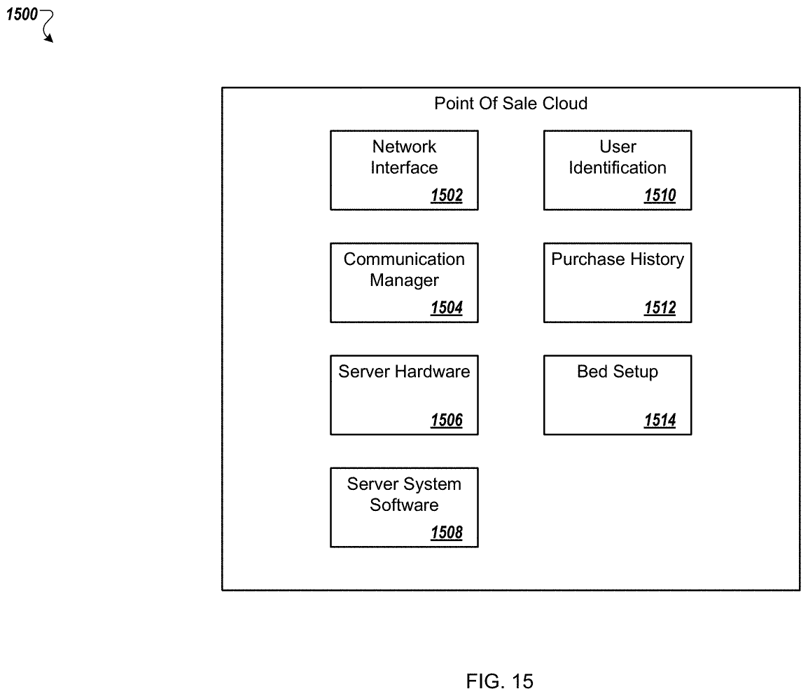

FIG. 15 is a block diagram of an example point of sale cloud service 1500 that can be used in a data processing system associated with a bed system, including those described above with respect to FIGS. 1-3. In this example, the point of sale cloud service 1500 is configured to record data related to users' purchases, specifically purchases of bed systems described herein.

The point of sale cloud service 1500 is shown with a network interface 1502, a communication manager 1504, server hardware 1506, and server system software 1508. In addition, the point of sale cloud service 1500 is shown with a user identification module 1510, a purchase history module 1512, and a bed setup module 1514.

The purchase history module 1512 can include, or reference, data related to purchases made by users identified in the user identification module 1510. The purchase information can include, for example, data of a sale, price, and location of sale, delivery address, and configuration options selected by the users at the time of sale. These configuration options can include selections made by the user about how they wish their newly purchased beds to be setup and can include, for example, expected sleep schedule, a listing of peripheral sensors and controllers that they have or will install, etc.

The bed setup module 1514 can include, or reference, data related to installations of beds that users purchase. The bed setup data can include, for example, a date and address to which a bed is delivered, a person who accepts delivery, configuration that is applied to the bed upon delivery (e.g., firmness settings), name or names of a user or users who will sleep on the bed, which side of the bed each user will use, etc.

Data recorded in the point of sale cloud service 1500 can be referenced by a user's bed system at later dates to control functionality of the bed system and/or to send control signals to peripheral components according to data recorded in the point of sale cloud service 1500. This can allow a salesperson to collect information from the user at the point of sale that later facilitates automation of the bed system. In some examples, some or all aspects of the bed system can be automated with little or no user-entered data required after the point of sale. In other examples, data recorded in the point of sale cloud service 1500 can be used in connection with a variety of additional data gathered from user-entered data.

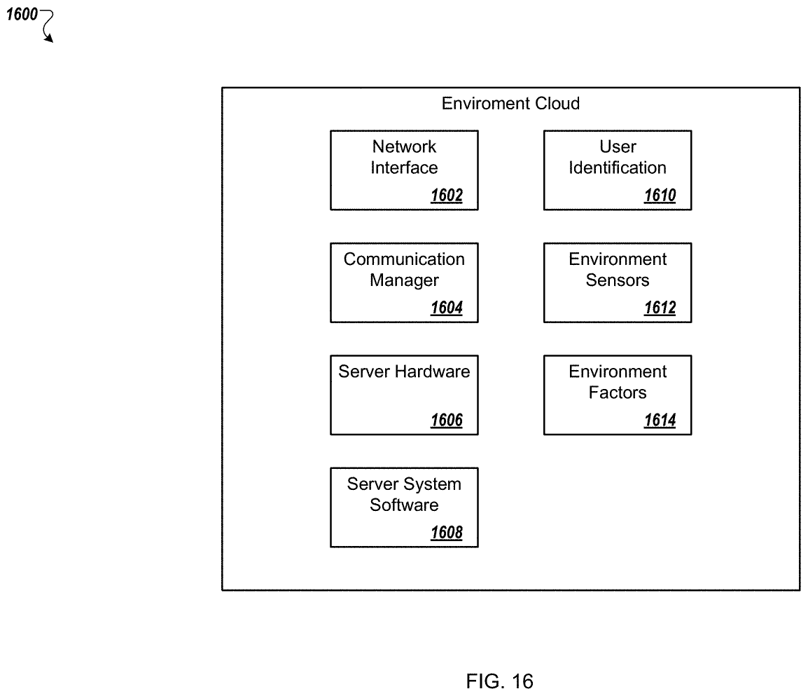

FIG. 16 is a block diagram of an example environment cloud service 1600 that can be used in a data processing system associated with a bed system, including those described above with respect to FIGS. 1-3. In this example, the environment cloud service 1600 is configured to record data related to users' home environment.

The environment cloud service 1600 is shown with a network interface 1602, a communication manager 1604, server hardware 1606, and server system software 1608. In addition, the environment cloud service 1600 is shown with a user identification module 1610, an environmental sensors module 1612, and an environmental factors module 1614.

The environmental sensors module 1612 can include a listing and identification of sensors that users identified in the user identification module 1610 have installed in and/or surrounding their bed. These sensors may include any sensors that can detect environmental variables, including but not limited to light sensors, noise/audio sensors, vibration sensors, thermostats, movement sensors (e.g., motion), etc. Additionally, the environmental sensors module 1612 can store historical readings or reports from those sensors. The environmental sensors module 1612 can then be accessed at a later time and used by one or more of the cloud services described herein to determine sleep quality and/or health information of the users.

The environmental factors module 1614 can include reports generated based on data in the environmental sensors module 1612. For example, the environmental factors module 1614 can generate and retain a report indicating frequency and duration of instances of increased lighting when the user is asleep based on light sensor data that is stored in the environment sensors module 1612.

In the examples discussed here, each cloud service 410 is shown with some of the same components. In various configurations, these same components can be partially or wholly shared between services, or they can be separate. In some configurations, each service can have separate copies of some or all of the components that are the same or different in some ways. Additionally, these components are only provided as illustrative examples. In other examples, each cloud service can have different number, types, and styles of components that are technically possible.

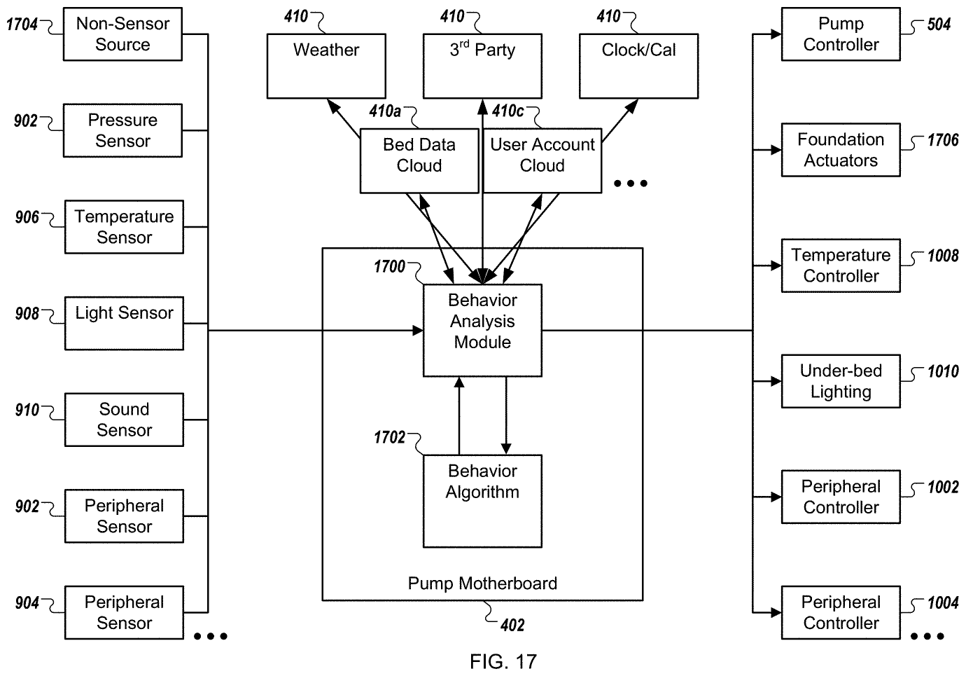

FIG. 17 is a block diagram of an example of using a data processing system associated with a bed (e.g., a bed of the bed systems described herein, such as in FIGS. 1-3) to automate peripherals around the bed. Shown here is a behavior analysis module 1700 that runs on the pump motherboard 402. For example, the behavior to analysis module 1700 can be one or more software components stored on the computer memory 512 and executed by the processor 502.

In general, the behavior analysis module 1700 can collect data from a wide variety of sources (e.g., sensors 902, 904, 906, 908, and/or 910, non-sensor local sources 1704, cloud data services 410a and/or 410c) and use a behavioral algorithm 1702 (e.g., one or more machine learning models) to generate one or more actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The behavior analysis module 1700 can collect data from any technologically appropriate source, for example, to gather data about features of a bed, the bed's environment, and/or the bed's users. Some such sources include any of the sensors of the sensor array 406 that is previously described (e.g., including but not limited to sensors such as 902, 904, 906, 908, and/or 910). For example, this data can provide the behavior analysis module 1700 with information about a current state of the environment around the bed. For example, the behavior analysis module 1700 can access readings from the pressure sensor 902 to determine the pressure of an air chamber in the bed. From this reading, and potentially other data, user presence in the bed can be determined. In another example, the behavior analysis module 1700 can access the light sensor 908 to detect the amount of light in the bed's environment. The behavior analysis module 1700 can also access the temperature sensor 906 to detect a temperature in the bed's environment and/or one or more microclimates in the bed. Using this data, the behavior analysis module 1700 can determine whether temperature adjustments should be made to the bed's environment and/or components of the bed in order to improve the user's sleep quality and overall comfortability.

Similarly, the behavior analysis module 1700 can access data from cloud services and use such data to make more accurate determinations of user sleep quality, health information, and/or control of the user's bed and/or peripheral devices. For example, the behavior analysis module 1700 can access the bed cloud service 410a to access historical sensor data 1212 and/or advanced sleep data 1214. Other cloud services 410, including those previously described can be accessed by the behavior analysis module 1700. For example, the behavior analysis module 1700 can access a weather reporting service, a 3^{rd} party data provider (e.g., traffic and news data, emergency broadcast data, user travel data), and/or a clock and calendar service. Using data that is retrieved from the cloud services 410, the behavior analysis module 1700 can more accurately determine user sleep quality, health information, and/or control of the user's bed and/or peripheral devices.

Similarly, the behavior analysis module 1700 can access data from non-sensor sources 1704. For example, the behavior analysis module 1700 can access a local clock and calendar service (e.g., a component of the motherboard 402 or of the processor 502). The behavior analysis module 1700 can use the local clock and/or calendar information to determine, for example, times of day that the user is in the bed, asleep, waking up, and/or going to bed.

The behavior analysis module 1700 can aggregate and prepare this data for use with one or more behavioral algorithms 1702. As mentioned, the behavioral algorithm 1702 can include machine learning models. The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

In the example shown, the behavioral analysis module 1700 and the behavioral algorithm 1702 are shown as components of the pump motherboard 402. However, other configurations are possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

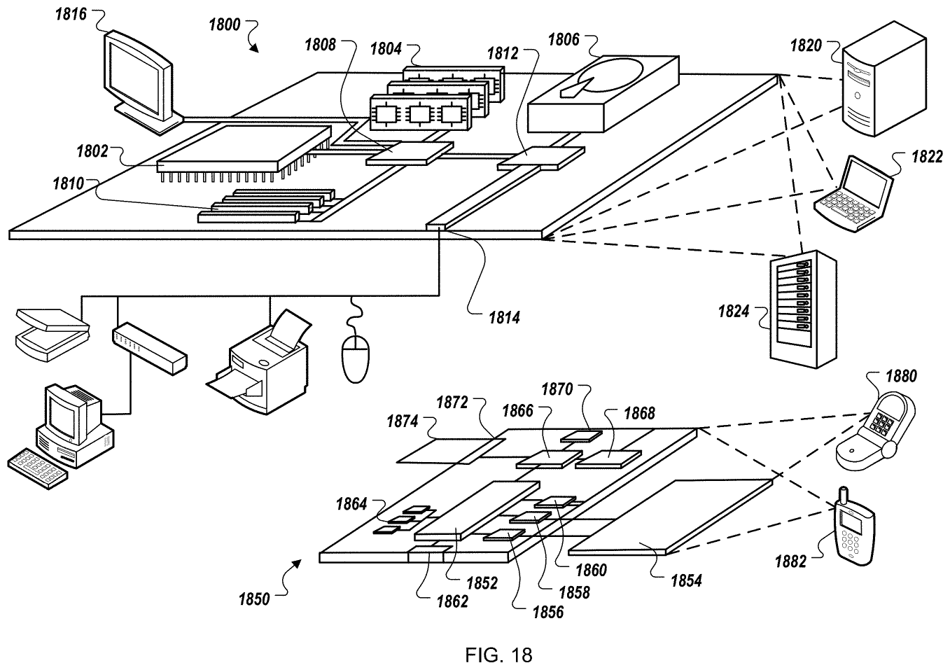

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk.

The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other.

The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850.

The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850.

The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850.

The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

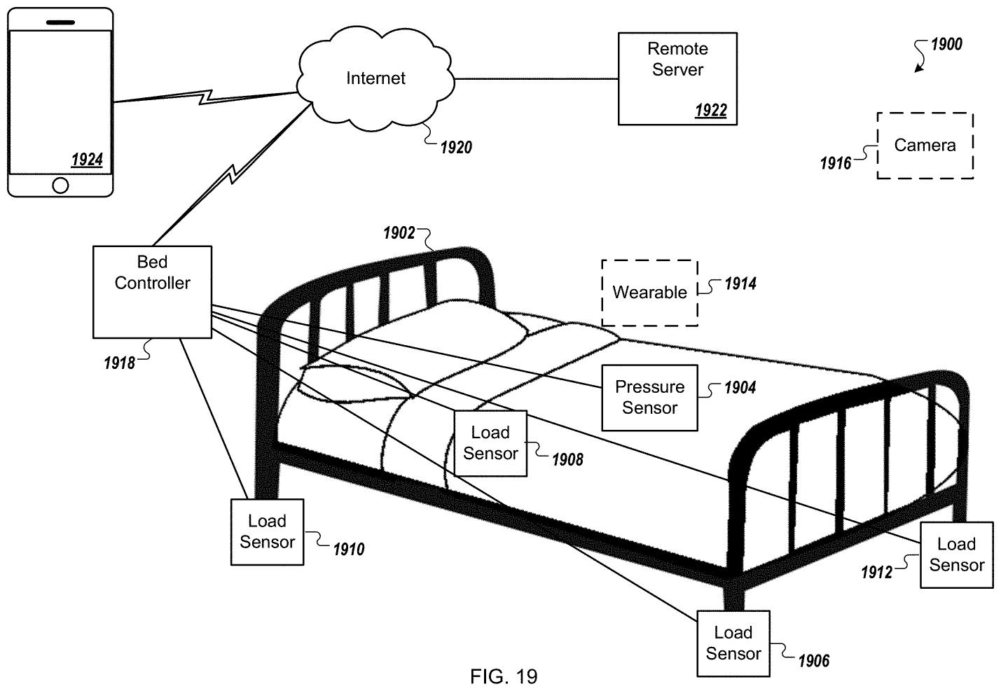

FIG. 19 is a diagram of a system 1900 with a bed used to passively monitor blood pressure. In the system 1900, one or more sensors integrated into, or used in conjunction with, the bed are used to collect balistocardiograph (BCG) data from at least two portions of a user's body. This user can be laying on the bed and may be asleep, but need not necessarily be asleep. For example, the user may lay on the bed specifically to check their BP, also the user may lay in bed reading a book and the system 1900 can passively monitor the user's BP in that time as well.

The bed 1902 is shown as a single person's bed, as might be used by a person that sleeps alone or as might be used in a hospital or sleep-lab setting. However, it will be understood that other bed arrangements may be used, including a bed with two users, or a bed large enough for two sleepers but with only one person on the bed. Various sensors in the bed, around the bed in the environment, worn by a user, etc. collect BCG data from at least two areas of the user's body. The particulars of these sensors can vary depending on the hardware available and other consideration. For example, a setup with a bed with a single user may use sensors in all four legs of a bed to produce BCG data for the user, while another bed with four legs may use two legs for one user and the other two legs for the other user. Still another bed may have no legs and may use other types of sensors. While at least one sensor is in or associated with the bed, one or more other sensors may be separate from the bed.

In the example shown, a first BCG sensor 1904 is configured to collect first BCG data from a first location of the user laying on the bed due to pressure applied to the bed by the user as well as other pressure such as bedding or a pillow. In this case, the first BCG sensor 1904 is a pressure sensor 1904 that is integrated in the mattress of the bed 1900, in fluidic communication with an air bladder in or under the mattress, etc. such that cardiac activity in a central portion of the user's body (e.g., the torso) is measured by the pressure sensor 1904. Other types of sensors can be used that collect first BCG data due to pressure applied to the bed by the user. For example, a load sensor or other kind of sensor may be used to collect first pressure data.

In the example shown, a second BCG sensor 1906 is configured to collect BCG data from a second location of the user. In this case, the second BCG sensor 1906 is a load-cell that measures load through the leg of the bed due to pressure applied to the bed by the user as well as other load such as the mattress, bed frame, bedding, pillow, etc. In this case, the second BCG sensor 1906 is integrated into the bed, but other second BCG sensors need not be.

As will be appreciated, the pressure sensors 1904 and 1906 will differentially measure movement of the user based on where in the bed the pressure is applied. A pressure applied directly to the center of the mattress can be measured as a higher value than the same pressure collected by the load sensor 1906 due to the pressure being distributed across four legs. Similarly, the location of the pressure will affect the relative readings. A pressure near the corner of the bed above the load cell 1906 will show more agreement between the load cell 1906 and the pressure sensor 1904 while a pressure father away will show less agreement. In this way, the location of pressure can be determined by two or more sensors. As such, the location of a pressure wave in a user's circulatory system can be tracked as it moves from the heart, to the right foot.

More or different BCG sensors can be used. For example, load cell 1908-1912 can be used in conjunction with the load cell 1906 and 1904 to provide five BCG data streams that can be together to find BP of the user with greater accuracy, redundancy, or precision (e.g., to ensure enough sources of data to fully triangulate—as will be understood, full triangulation in 3D space uses at least four sensors).

Other types of sensors capable of collecting BCG data may be used. One or more wearable devices 1914, for example, can include a photoplethysmography (PPG) sensor capable of measuring cardiac activity in a given, constant location on the user's body such as the wrist for a watch, the torso for a chest-strap, the head for a head-worn electroencephalogram EEG sensor (including consumer-grade EEG sensors or medical-grade EEG sensors), the finger for a ring or pulse-ox sensor, etc. One or more cameras 1916 can collect energy emissions of the user's skin that are influenced by the movement of blood under the skin. In some cases, these cameras 1916 can be used with object-tracking processes to identify particular locations on a user's body such as the neck or forehead that are unlikely to be occluded by bedding or clothing. The camera 1916 can capture visible light and/or non-visible energy such as thermal energy generated by the user or reflected energy such as in a LIDAR or RADAR sensor.

A computer-system with processor and memory can be used to process the data from the sensors and generate BP values for the user can receive the BCG data from the sensors. In this example, a bed controller 1918 collects pressure raw data from the sensors 1904-1912 and performs some pre-processing operations such as checking data integrity, compressing or sampling the data, and marshaling the data into serialized data objects for transmission over a network like the Internet 1920. A remote server 1910 can receive the data from the bed controller 1918 and determine from the data one or more BP values. A user device 1924 can retrieve the BP values from the server 1922 and display the BP value. However, other configurations are possible. For example, the bed controller 1918 can determine the BP and report the BP to the remote server 1922 and/or user device 1924. In another example, the user device 1924 may receive the sensor values directly from the sensors 1904-1912 and determine the BP values.

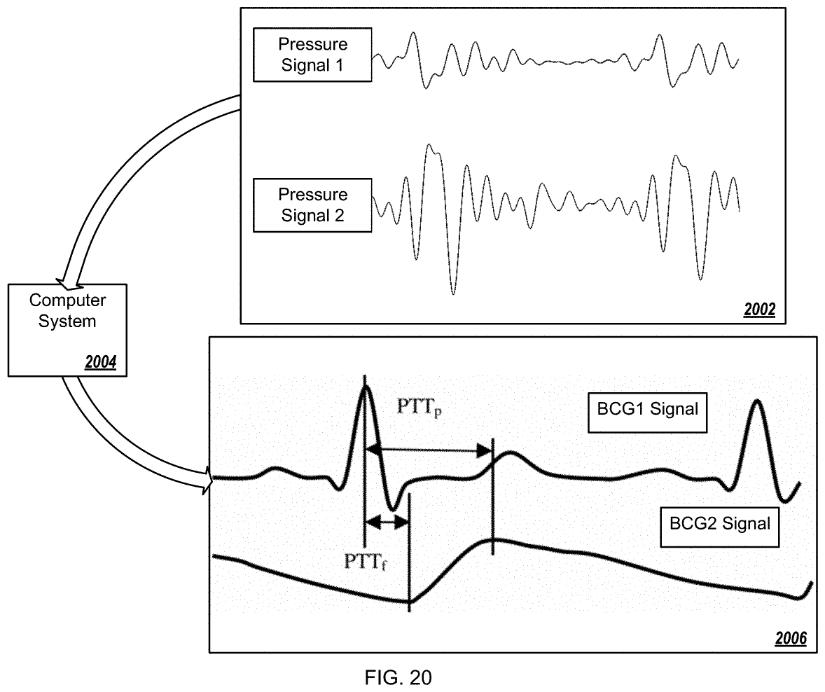

FIG. 20 shows elements of pressure signals used to determine pulse transit times. In this example, raw pressure signals 1 and 2 are shown in window 2002. A computer system 2004 can access these signals (e.g., as streams of binary data) and generate from them BCG signals 1 and 2 shown in window 2006 (e.g., as streams of binary data). While only two pressure signals and BCG signals are shown, it will be appreciate that different numbers (e.g., five for four load cells and a central pressure value) or types (e.g., from a different sensor such as a camera) may be used in other examples.

One or more PTT values may be determined by the computer system 2004. For example, the computer system 2004 may identify a spike in the BCG1 signal based on a matching of a mathematical or logical test (e.g., a local maximum preceded by and followed by rates of change greater than a given threshold). Then, subsequent features in the BCG2 signal can be found and time differences recorded. In this example, a PTT_{f} is found to a trough point in the BCG2 signal and a PTTₚ is found to a peak point in the BCG2 signal. One, both, a combination, or other similar PTT time can be used by the computer system 2004 to determine BP, as will be described elsewhere in this document.

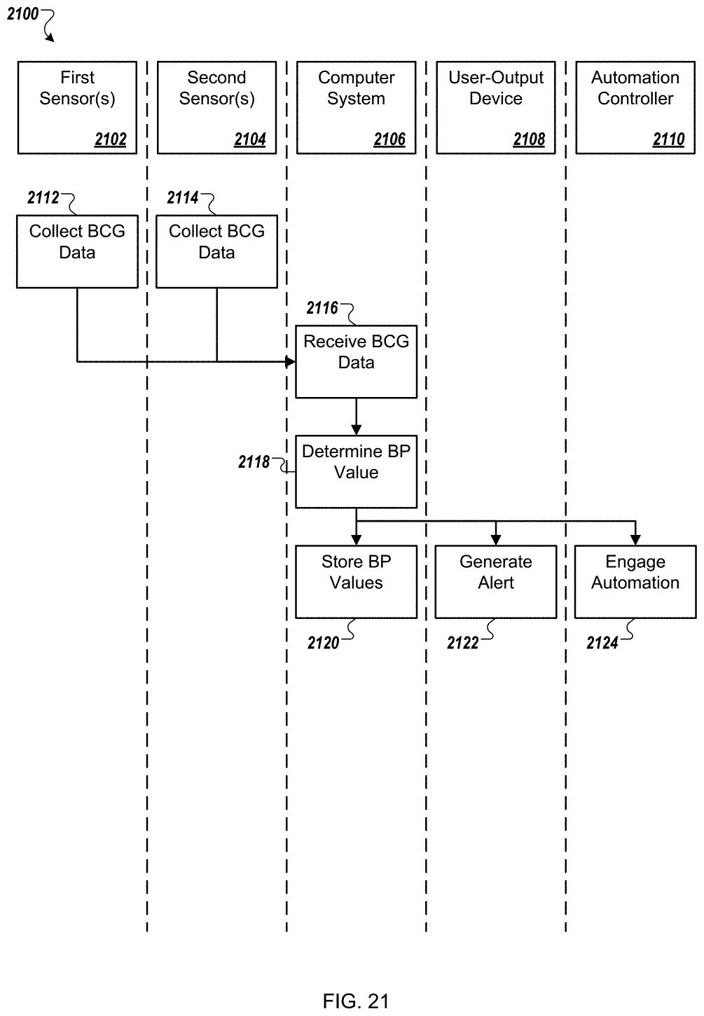

FIG. 21 is a swimlane diagram of an example process 2100 for determining blood pressure values. For clarity, the process 2100 is being described with reference to particular components of sensors 2102 and 2104, a computer system 2106, a user-output device 2108, and an automation controller 2110. However, other system or systems can be used to perform the same or a similar process.

The first sensor 2102 collects first BCG data 2112 and transmits the first BCG data to the computer system. The second sensor 2104 collects second BCG data 2114 and transmits the second BCG data to the computer system 2106. For example, a the first sensor 2102 may sense pressure applied to an air bladder of the bed of the user, and may transmit that pressure data to the computer system while simultaneously the second sensor 2104 and three other load cells may each sense load being transmitted through a leg of the bed and may transmit that pressure data to the computer system.

The computer system 2106 receives the first BCG data and the second BCG data 2116. For example, a bed controller of the computer system 2106 can collect the first BCG data and the second BCG data and transmit the collected data to a remote server.

The computer system 2106 determines one or more blood-pressure (BP) values for the user 2118. For example, the remote server of the computer system 2106 can process the first BCG data and the second BCG data to generate BP values with associated timestamps for the user. Example processes of determination are described elsewhere in this document.

The computer system 2106 stores the BP value to memory 2106. For example, the computer system 2106 can log the BP values indexed by their timestamp so that, for example the computer system 2106 can respond to queries requesting BP values for the use within a particular time window or other queries related to or unrelated to time.

The user-output device 2108 generates an alert for output to a user-output device 2108. For example, the computer system 2106 may maintain a ruleset that defines dangerous BP ranges for all users, for a specific user, or for classes of users (e.g, groups defined by demographic information, specification of an illness or risk factor, user opt-in). When BP values are detected in the unsafe range (e.g., greater or less than a given threshold, above or below another threshold for a certain amount of time), an alert can be generated and output by a user device such as a mobile phone, bedside health hub, or nurse's console. In some cases, graduated risk bands are defined that define progressively greater risk, with different alerts for each. For example, an elevated BP that poses a chronic but not acute risk can be defined to generate an email, while a very low BP that poses an acute risk can define an alert to generate a loud alarm to awaken the user and draw their attention or alert a medical provider.

The automation controller 2110 engages an automation device 2124. For example, the HVAC or environmental conditions may be modified by an HVAC or fan controller to change the temperate of the sleeping environment around the user based on a BP value out of an ideal or goal range. In another example, a morning alarm system may wake the user at a time based on their BP, with an earlier alarm being enabled to give the user more gym time in the morning to work on managing hypertension. One type of particularly advantageous implementation involves the modulation of the microclimate—the temperature directly around the skin of the user in the bed. For example, heating elements in the bed and/or bedding can be engaged to increase the temperature of the microclimate, which is believed to facilitate blood flow via vasodilation which may decrease BP of the user while the user is sleeping, which may reduce their risk of adverse health events.

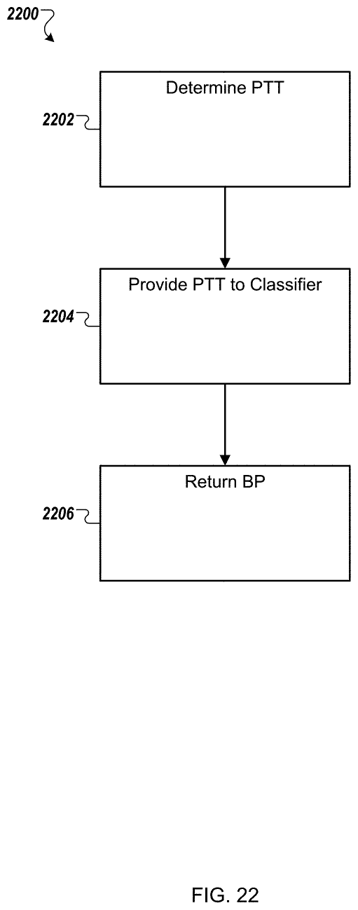

FIG. 22 is a flowchart of an example process 2200 for determining blood pressure values. For example, the process 2200 can be performed by the computer system 2106 when determining BP values 2118.

One or more PTTs are determined for the user of the bed 2202. The PTT defined the length of time between when a pressure wave in a circulatory system reaches one point and then reaches another point. Various types of PTT metrics are possible, each one defining a different test or tests to be applied to two or more BCG data streams.

In some cases, more than two data streams are available. For example, in the case of a bed with an air bladder and four legs with load cells, the streams may be labeled BCG₀ for the air bladder and BCG₁, BCG₂, BCG₃ and BCG₄ for each of the four load cell streams. In this case, a unified PTT_{proxy} value may be found by combining a PTT found for every combination of BCG₀ and another BCG stream resulting in a PTT₁, PTT₂, PTT₃, and PTT₄. Said another way, one BCG stream may be treated as a central or base BCG and the other BCG values may be treated as peripheral BCG values that can be combined. In some cases this combination may be a simple arithmetic average of PTT₁₋₄, but other combinations are possible. For example, the highest and lowest PTTs may be excluded to avoid using momentary missreadings, which is a possibility when, for example, a sensor is damaged and will only return maximum and minimum values.

The PTT or PTTs used is provided as input to a BP classifier 2204. The classifier can take the form of a software object or objects that include a function that process the input according to a model stored in computer memory. This model may logically and mathematically correlate input PTT values with corresponding BP values. In many, but not all cases, these models can be generated from analysis and/or computer learning processes created with a training data of example PTT values and BP values collected with another sensing modality. For example, the training data may be created by having users lay on a bed with the BCG sensors while their BP is collected using an arm-worn cuff. By using timestamp data, events in the BCGs—as defined by their PTT—can be paired with BP readings at the same time. Then, with this training data, the model or models used by the classifiers can be generated.

A variety of different types of models may be used alone or in combinations with each other. In some cases, the BP classifiers have been trained with a linear model finding a fit between training-PTT values and training-BP values. For example, in the example with PTT₁₋₄ described above, a linear model in the following form may be used:

BP=k₀+k₁*PTT₁+k₂*PTT₂+k₃*PTT₃+k₄*PTT₄

Then, processes such as linear regression can be used to find the values k₀₋₄ that minimize the difference between the predicted BP values according to the equation given PTT values versus the actual measures BP values associated with the PTT values.

In some cases, the BP classifiers have been trained using a polynomial model finding a fit between training-PTT values and training-BP values such as a quadratic or cubic regression. For example, in the example with PTT₁₋₄ described above, a quadratic model in the following form may be used:

$$BP = k 0 + {\sum }_{i = 1}^{4} {k}_{i} {PTT}_{i} + {\sum }_{i , j = 1}^{4} {k}_{i , j} {PTT}_{i} {PTT}_{j}$$

$$BP =$$

Then, processes such as quadratic regression can be used to find the values k₀₋₆ that minimize the difference between the predicted BP values according to the equation given PTT values versus the actual measures BP values associated with the PTT values.

In some cases, processes such as boosted decision tree regression can be used to find relationships between training-BP values and training-PTT values (or other training data). In some implementations, PTT values are used as feature vectors to be processed by regression trees to estimate BP values. An example of a boosted decision tree analysis uses a maximum depth of ten, a learning rate of 0.05, 500 estimators, an L2 regularization set to a value of one, and an L1 regularization set to a value of zero, though other configurations may be used. One possible advantage of the use of boosted decision trees is that the importance of each feature in the feature vector can be determined by analyzing the relative frequency of the use of each feature in the branches of the boosted decision tree, with more common use indicating greater importance in the analysis and thus in the classifier. This may be useful, for example, when computational resources are insufficient for full data capture and the more important features can be prioritized in the sensing, transmission, and storage operations of the system.

In some cases, the BP classifiers have been trained using a machine learning process that performs operations to generate a data structure that generates predicted BP values for given input PTT values. One example of such a data structure is a neural network such as a convolutional neural network that defines nodes organized into layers and finds weight values to assign to edges between nodes of adjacent layers.

The classifier returns the BP value and the BP values for the user are received as output 2206. This output can include, or be annotated with, relevant metadata such as a timestamp of the calculation, a timestamp of the peak identified in the PTT₀, etc. The metadata an also include the version number of the classifier and the PTT₀₋₄ used to allow for old BP values can be updated when a new version of a classifier is developed, etc.

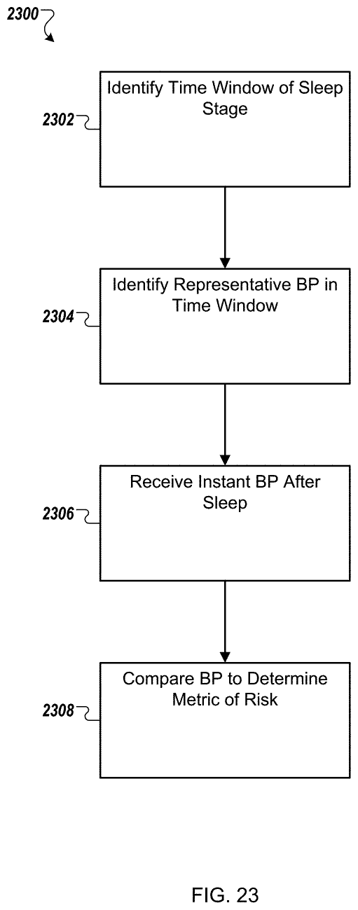

FIG. 23 is a flowchart of an example process 2300 for determining a metric of risk based on blood pressure values. The process 2300 may be used, for example, after BP is determined 2118 and BP values are stored 2120 and/or to generate an alert 2122.

It is believed that a comparison of nighttime BP and morning BP can be used as a metric of risk of, for example, of cardiovascular disease (CVD) risk. This process describes some way in which a nighttime BP and morning BP can be compared, but it will be appreciated that variations are possible. For example, this process will describe use of a nighttime BP from a first sleep stage in the sleep session, but a nighttime BP from the entire sleep session or another portion of the sleep session may be used.

A time window of a particular sleep stage of a sleep session of the user sleeping on the bed is identified 2302. For example, when the user enters the bed, the user's biometrics may be monitored for the onset of sleep. Then, the next 90 minutes from the onset of sleep may be identified as the time window of the first sleep session based on an understanding that 90 minutes is a generally useful approximation of a sleep stage for most users. However, the sleep stage can also be determined from biometric information collected such as breath and cardiac action that can be used to determine sleep stage.

A representative-BP for the sleep stage is identified based on the one or more BP values for the user which are within the time window 2304. For example, the BP values may be combined and filtered to find particular BP values or particular proxy BP values fitting a particular defining. In some cases, a minimum or near-minimum value may be found so as to identify the lowest BP of the user in the sleep stage.

One process for finding this representative-BP is to select the N^{th} lowest BP value within the time window to represent a low-BP value for the sleep stage (e.g., the tenth lowest). One process for finding this representative-BP is to select the N^{th} lowest percentile BP value within the time window to represent a low-BP value for the sleep stage (e.g. the 10^{th} percentile value). By use of these types of processes, outlier values may be excluded that are not accurate readings—for example, due to sensor fault or anomalous user movement.

An instant-BP value is received for the user taken after the sleep session 2306. For example, the user may be instructed to wake up after the sleep session, being their morning grooming routine, and then take their BP once they feel awake.

To generate this instant-BP value, the user may use a different source such as an inflatable cuff or other device different than the bed that the user slept in. In some cases, the user may lay back on the bed to generate the instant-BP value. As has been described, the technology described in this document does not require the user to sleep in order for a BP value to be determined.

The low-BP value for the sleep stage is compared with the instant-BP value 2308 to determine a BP-dip value for the user representing an amount of dip in BP the user demonstrates in the sleep session as it is believed that the dip will mostly occur during the first part of sleep such as within the first two sleep cycles, which may be within four hours of sleep onset. This dip may be a measure of the difference between the sleeping BP and waking BP value. It is believed that this dip can be used as an indication of risk for the user, for example with a greater than a given threshold (e.g., 20%) associated with increased risk for ischemic stroke and silent cerebral disease and a dip less than another threshold (e.g., 10%) or an increase creating an inverse-dip being associated with increased risk of death, myocardial infarction, and stroke.

Responsive to determining the BP-dip value, actions can be performed using the BP-dip value such as storing the BP-dip to the memory, generating an alert for output to a user-output device, engaging an automated device, as has been described in the process 2200.

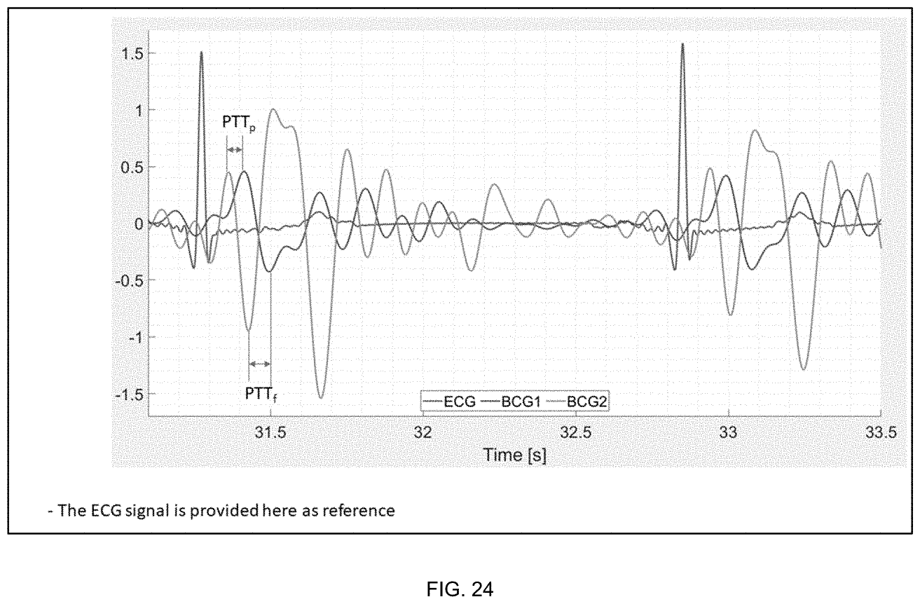

FIG. 24 shows an example of two BCGs captured and plotted against a directly measured ECG, which may be used, for example, as training data to create one or more models.

In another embodiment, a generic model to estimate BP from PTT values is trained using a large dataset that comprises ground-truth data from several individuals. The generic model is then personalized using small calibration data (e.g. one-minute long calibration sequence with known BP values with a cuff-based device and PTT values extracted from the bed). Such personalization can be easily accomplished with a decision tree type of model as this type of models can be adjusted to accommodate new training data. This process is known as “transfer learning”.

What is claimed is:

1. A system comprising:
a bed having a mattress to support a user laying on the bed;
a first balistocardiograph (BCG) sensor configured to collect first-BCG data from a first location of the user laying on the bed due to pressure applied to the bed by the user;
a second BCG sensor configured to collect second-BCG data from a second location of the user;
a computer-system comprising a process and memory, the computer-system configured to:
receive the first BCG-data and the second-BCG data; and
determine one or more blood-pressure (BP) values for the user.

2. The system of claim 1, wherein the first BCG sensor is a pressure sensor configured to sense pressure readings applied to the bed by the user due to weight and motion of the user.

3. The system of claim 2, wherein the first BCG sensor is one of the group consisting of a pressure transducer and a load cell.

4. The system of claim 1, wherein the second BCG sensor is a system comprising one or more load cells configured to sense load applied to a support member of the bed by the user due to weight and motion of the user.

5. The system of claim 1, wherein the second BCG sensor is a device worn by the user on a specified location of the user's body.

6. The system of claim 1, wherein the second BCG sensor is a camera configured to sense energy emissions of the user's skin, the energy emissions being one of the group consisting of visible light and non-visible energy.

7. The system of claim 1, wherein:
the first BCG sensor is a pressure sensor configured to sense pressure readings applied to the bed by the user due to weight and motion of the user;
the first BCG sensor is one of the group consisting of a pressure transducer and a load cell; and
the second BCG sensor is a system comprising one or more load cells configured to sense load applied to a support member of the bed by the user due to weight and motion of the user.

8. The system of claim 1, wherein to determine one or more BP values for the user, the computer-system is configured to:
determine a pulse transit time (PTT) for the user identifying a length of time between a pulse event in the first BCG data and the second BCG data representing the length of time between when a pulse of the user's blood reaches the first location and the second location;
providing, as input, the PTT to a BP classifier; and
receiving, as output, the one or more blood-pressure values for the user.

9. The system of claim 8, wherein the BP classifier has been trained using at least one model of the group consisting of:
i) a linear model finding a fit between training-PTT values and training-BP values;
ii) a polynomial model finding coefficients describing a function that relates the training-PTT values with the training-BP value;
iii) a machine-learning model that creates a data-structure created by a machine-learning process; and
iv) a boosted decision tree regression model using the training-PTT values a feature vectors.

10. The system of claim 9, wherein the data structure is a convolutional neural network.

11. The system of claim 1, wherein the computer-system is further configured to:
identify a time window of a particular sleep stage of a sleep session of the user sleeping on the bed;
identify a representative-BP for the sleep stage based on the one or more blood-pressure (BP) values for the user which are within the time window.

12. The system of claim 11, wherein the representative-BP is the N^{th} lowest BP value within the time window to represent a low-BP value for the sleep stage.

13. The system of claim 11, wherein the representative-BP is the N^{th} lowest percentile BP value.

14. The system of claim 11, wherein the computer-system is further configured to:
receive an instant-BP value for the user taken after the sleep session;
comparing the low-BP value for the sleep stage with the instant-BP value to determine a BP-dip value for the user representing an amount of dip in BP the user demonstrates in the sleep session.

15. The system of claim 13, wherein the computer system is further configured to, responsive to determining the BP-dip value, perform, using the BP-dip value, at least one of the group consisting of: storing the BP-dip to the memory, generating an alert for output to a user-output device, engaging an automated device.

16. The system of claim 11, wherein the time window is between 0 and 4 hours after sleep onset.

17. The system of claim 1, wherein the computer-system is further configured to, responsive to determining the BP value, perform, using the BP value, at least one of the group consisting of: storing the BP value to the memory, generating an alert for output to a user-output device, engaging an automation device.

18. The system of claim 1 where a generic model is trained from a large dataset containing ground-truth data from several individuals and a personalized, more accurate, model is derived from the generic one by transfer learning the generic model using a small calibration dataset from the subject for whom the personalization process is conducted.

19. A system comprising:
one or more processors; and
computer memory storing instructions that, when executed by the one or more processors, cause the system to perform operations comprising:
receiving i) first-BCG from a first BCG sensor configured to collect the first-BCG data from a first location of a user laying on a bed due to pressure applied to the bed by the user, and ii) second-BCG from a second BCG sensor configured to collect the second-BCG data from a second location of the user; and
determining one or more BP values for the user.

20. A method comprising:
receiving i) first-BCG from a first BCG sensor configured to collect the first-BCG data from a first location of a user laying on a bed due to pressure applied to the bed by the user, and ii) second-BCG from a second BCG sensor configured to collect the second-BCG data from a second location of the user; and
determining one or more BP values for the user.
